(19) United States
(12) Patent Application Publication
(10) Pub. No.: US 2012/0191153 A1
(43) Pub. Date: Jul. 26, 2012

(54) DIAGNOSIS OF LEAD FRACTURE AND CONNECTION PROBLEMS

(75) Inventors: Charles D. Swerdlow, Los Angeles, CA (US); Haresh G. Sachanandani, Culver City, CA (US); Bruce D. Gunderson, Plymouth, MN (US)

(73) Assignee: Medtronic, Inc., Minneapolis, MN (US)

(21) Appl. No.: 13/011,559

(22) Filed: Jan. 21, 2011

(51) Int. Cl. A61N 1/37; G01R 27/28; A61N 1/39

(52) U.S. Cl. 607/8; 607/28; 324/649

(57) ABSTRACT

Techniques for diagnosing lead fractures and lead connection problems are described. One or more medical leads may be coupled to an implantable medical device (IMD) to position electrodes or other sensors at different locations within a patient than the IMD. The IMD may include a lead diagnostic module configured to diagnose problems with a coupled lead and automatically select between a lead fracture problem and a lead connection problem based on the diagnosis. The diagnosis of either lead fracture problems or lead connection problems may be based on a timing of an increased impedance value with respect to connection of the lead to the IMD, a return to baseline impedance values after the increased impedance value, an abrupt rise of the increased impedance value, maximum impedance values, or oversensing. An external device may present the diagnosis to a user to facilitate appropriate corrective action.

### TECHNICAL FIELD

The disclosure relates to implantable medical devices, and, more particularly, to evaluating integrity of an implantable medical device.

### BACKGROUND

A variety of implantable medical devices for delivering a therapy and/or monitoring a physiological condition have been clinically implanted or proposed for clinical implantation in patients. Implantable medical devices may deliver electrical stimulation or fluid therapy and/or monitor conditions associated with the heart, muscle, nerve, brain, stomach or other organs or tissue. Some implantable medical devices may employ one or more elongated electrical leads carrying stimulation electrodes, sense electrodes, and/or other sensors. Implantable medical leads may be configured to allow electrodes or other sensors to be positioned at desired locations for delivery of stimulation or sensing. For example, electrodes or sensors may be carried at a distal portion of a lead. A proximal portion of the lead may be coupled, e.g., connected, to an implantable medical device housing, which may contain circuitry such as stimulation generation and/or sensing circuitry.

Implantable medical devices, such as cardiac pacemakers or implantable cardioverter-defibrillators, for example, provide therapeutic electrical stimulation to the heart via electrodes carried by one or more implantable leads. The electrical stimulation may include signals such as pulses for pacing, or shocks for cardioversion or defibrillation. In some cases, an implantable medical device may sense intrinsic depolarizations of the heart, and control delivery of stimulation signals to the heart based on the sensed depolarizations. Upon detection of an abnormal rhythm, such as bradycardia, tachycardia or fibrillation, an appropriate electrical stimulation signal or signals may be delivered to restore or maintain a more normal rhythm. For example, in some cases, an implantable medical device may deliver pacing pulses to the heart of the patient upon detecting tachycardia or bradycardia, and deliver cardioversion or defibrillation shocks to the heart upon detecting tachycardia or fibrillation.

Leads associated with an implantable medical device typically include a lead body containing one or more elongated electrical conductors that extend through the lead body from a connector assembly provided at a proximal lead end to one or more electrodes located at the distal lead end or elsewhere along the length of the lead body. The conductors connect stimulation and/or sensing circuitry within an associated implantable medical device housing to respective electrodes or sensors. Some electrodes may be used for both stimulation and sensing. Each electrical conductor is typically electrically isolated from other electrical conductors and is encased within an outer sheath that electrically insulates the lead conductors from body tissue and fluids.

Cardiac lead bodies tend to be continuously flexed by the beating of the heart. Other stresses may be applied to the lead body during implantation or lead repositioning. Patient movement can cause the route traversed by the lead body to be constricted or otherwise altered, causing stresses on the lead body. The electrical connection between implantable medical device connector elements and the lead connector elements can be intermittently or continuously disrupted. Connection mechanisms, such as set screws, may be insufficiently tightened at the time of implantation, followed by a gradual loosening of the connection. Also, lead pins may not be completely inserted into the corresponding implantable medical device connector elements. In some cases, changes in leads or connections may result in intermittent or continuous changes in lead impedance.

Short circuits, open circuits or significant changes in impedance may be referred to, in general, as lead related conditions. In the case of cardiac leads, sensing of an intrinsic heart rhythm through a lead can be altered by lead related conditions. Structural modifications to leads, conductors or electrodes may alter sensing integrity. Furthermore, impedance changes in the stimulation path due to lead related conditions may affect sensing and stimulation integrity for pacing, cardioversion, or defibrillation. In addition to lead related conditions, conditions associated with sensor devices or sensing circuitry may affect sensing integrity.

### SUMMARY

In general, this disclosure describes techniques for diagnosing lead fractures and lead connection problems, i.e., problems with the connection between a lead and an implantable medical device. Leads may be implanted within a patient and coupled to an implantable medical device (IMD). Once implanted, however, correctly diagnosing problems with a lead may be difficult. These problems may include, for example, fractures of one or more lead wires within the lead or incomplete connections between a lead connector and a header of the IMD. As further described herein, the IMD and/or an external device may automatically differentiate, or distinguish, between types of lead problems, and present the diagnosis to a clinician or other healthcare professional. This differentiation between lead connection problems and lead fracture problems may avoid unnecessary explantation of non-fractured leads. Accordingly, leads diagnosed with a lead connection problem may be simply reconnected to the IMD header.

The diagnosis of either a lead fracture or a lead connection problem may be based on one or more of impedance, the timing of impedance changes, or oversensing characteristics of the lead. The IMD coupled to the lead may periodically measure an impedance of the lead. Certain characteristics of the impedance may be analyzed to diagnose problems with the lead or its connections to the IMD. For example, the diagnosis of either lead fracture or a lead connection problem may be based on a timing of an increased impedance value with respect to when the lead was connected to the IMD, the timing of a return, if any, to a baseline or near-baseline impedance value after the increased impedance value is detected, a maximum impedance value, or oversensing of cardiac events in the electrical signal, e.g., cardiac electrogram, monitored via the leads. An external device, e.g., a clinician programmer, may present the diagnosis to a user to facilitate appropriate corrective action.

In one example, the disclosure describes a method that includes measuring a plurality of impedance values of an implantable medical lead, comparing each of the impedance values to a threshold, identifying at least one of the plurality of impedance values greater than the threshold as an increased impedance value, determining a timing of the increased impedance value, and automatically selecting between a diagnosis of a lead fracture or a diagnosis of a lead connection problem based on the timing of the increased impedance value.

In another example, the disclosure describes a system that includes an implantable medical device that measures a plurality of impedance values of an implantable medical lead coupled to the implantable medical device and a lead diagnostic module. The lead diagnostic module is configured to compare each of the impedance values to a threshold, identify at least one of the plurality of impedance values greater than the threshold as an increased impedance value, determine a timing of the increased impedance value, and automatically select between a diagnosis of a lead fracture or a diagnosis of a lead connection problem based on the timing of the increased impedance value.

In another example, the disclosure describes a system that includes means for measuring a plurality of impedance values of an implantable medical lead, means for comparing each of the impedance values to a threshold, means for identifying at least one of the plurality of impedance values greater than the threshold as an increased impedance value, means for determining a timing of the increased impedance value, and means for automatically selecting between a diagnosis of a lead fracture or a diagnosis of a lead connection problem based on the timing of the increased impedance value.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual drawing illustrating an example system configured to automatically diagnose lead fractures and lead connection problems, the system including a medical lead coupled to an implantable medical device (IMD).

FIG. 2A is a conceptual drawing illustrating the example IMD and leads of FIG. 1 in conjunction with a heart.

FIG. 2B is a conceptual drawing illustrating the example IMD of FIG. 1 coupled to a different configuration of implantable medical leads in conjunction with a heart.

FIG. 3 is a functional block diagram illustrating an example configuration of the IMD of FIG. 1.

FIG. 4 is a functional block diagram illustrating an example configuration of an external programmer that facilitates user communication with the IMD of FIG. 1.

FIG. 5 is a block diagram illustrating an example system that includes an external device, such as a server, and one or more computing devices that are coupled to the IMD and programmer shown in FIG. 1 via a network.

FIGS. 6A and 6B are conceptual illustrations of example complete and incomplete connections of a medical lead connector within a header of the IMD of FIG. 1.

FIG. 7 illustrates an example graph of impedance values measured over time from a lead diagnosed with a lead connection problem.

FIG. 8 illustrates an example graph of impedance values measured over time from a lead diagnosed with a lead fracture.

FIG. 9 is a flow diagram of an example method for diagnosing lead fractures and lead connection problems.

### DETAILED DESCRIPTION

This disclosure generally describes techniques for diagnosing lead fractures and connection problems that may arise between leads and implantable medical devices (IMDs). Medical leads generally include one or more conductive wires that are insulated from patient tissues and provide an electrical connection between one or more electrodes at the distal end of the lead and an IMD. After implantation of the lead, abnormal impedances or electrical signals may be detected from the lead. These abnormal impedances or signals may be caused by, for example, fractures of a wire within the lead (a lead fracture) or an incomplete connection between the IMD and a connector pin of the lead. An incomplete connection, or connection problem, may include a lead pin only partially inserted into the IMD header or a less than full tightening of the set screw such that the lead pin does not make a complete electrical connection with the IMD. Over time, lead bending and stretching may occur with patient movement to fracture the lead and/or partially disconnect the lead from the IMD.

Correctly distinguishing between an incomplete connection between the IMD and the lead or a lead fracture based on an analysis of the electrical signals may be difficult. Since signal variations caused by a lead connection problem may be similar to signal variations caused by lead fractures, non-fractured leads may be unnecessarily removed from the patient. Therefore, the patient may be subjected to explanation of the current lead and implantation of a replacement lead instead of a simpler procedure to correctly connect the lead with the IMD.

As described herein, the IMD and/or an external device may automatically differentiate between types of lead problems, e.g., connection problems and lead fracture problems, and present the diagnosis to a clinician or other healthcare professional. This differentiation between lead connection problems and lead fracture problems may avoid unnecessary explantation of non-fractured leads. Accordingly, a clinician may simply reconnect a lead to the IMD header if the diagnosis indicates a lead connection problem. Although this diagnosis may be referred to as a type of lead integrity analysis, the integrity of the lead and the integrity of the connection between the IMD are both being analyzed.

The diagnosis of either a lead fracture or a lead connection problem may be based on one or more of impedance, the timing of impedance changes, or oversensing characteristics from the lead. In general, oversensing may include the sensing of any signals other than an anticipated or desired R-wave or P-wave, depending on lead being used to sense the electrical signals. Oversensing may also include erratic noise in an electrogram or saturation that may occur with a lead fracture or a connection problem that would not be present in the electrogram from a non-fractured lead with a complete connection to the IMD.

The IMD coupled to the lead may periodically measure an impedance of the lead. Certain characteristics of the impedance may be analyzed to diagnose any problems with the lead. For example, the diagnosis of either lead fracture or a lead connection problem may be based on a timing of an increased impedance value with respect to when the lead was connected to the IMD, the timing of a return, if any, to a baseline or near-baseline impedance value after the increased impedance value is detected, a maximum impedance value, or oversensing of events in the electrical signal by the IMD, e.g., cardiac electrogram, monitored via the leads.

The diagnosis may be delivered to a user via a variety of external devices. For example a clinician programmer may present the diagnosis to the user. In another example, a networked computer may present the diagnosis to the user. In some examples, the external device may generate the diagnosis, while in others the external device may receive the diagnosis from the IMD or a different external device, and present the diagnosis to the user. In some examples, a user may receive the diagnosis while located remotely from the patient, e.g., via a computer network. The communication of the diagnosis, or information from which the diagnosis may be derived, from the IMD may be user-requested or IMD-initiated. In some cases, the communication of a diagnosis may be in the form of an alarm notification. In any case, one or more devices may be configured to generate the diagnosis and/or present the diagnosis to a user, as described herein.

Although the techniques described herein are generally directed to cardiac leads, lead problem diagnosis may be performed on any type of electrical lead. For example, these diagnosis techniques may be used to diagnose problems with neurostimulation or subcutaneous leads used to deliver stimulation and/or monitor a physiological condition of the patient.

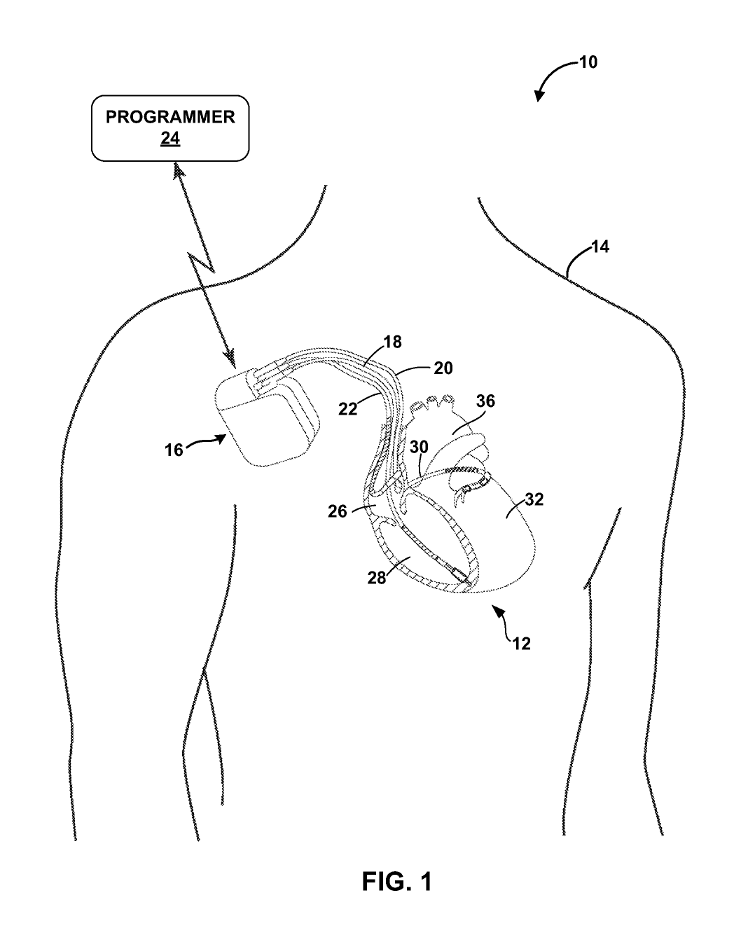

FIG. 1 is a conceptual drawing illustrating example system 10 configured to automatically diagnose lead fractures and lead connection problems. In the example of FIG. 1, system 10 includes IMD 16, which is coupled to leads 18, 20, and 22, and programmer 24. IMD 16 may be, for example, an implantable pacemaker, cardioverter, and/or defibrillator that provides electrical signals to heart 12 via electrodes coupled to one or more of leads 18, 20, and 22. Patient 14 is ordinarily, but not necessarily a human patient.

Although an implantable medical device and delivery of electrical stimulation to heart 12 are described herein as examples, the techniques for diagnosing lead fractures and lead connection problems between IMD 16 and any of leads 18, 20, and 22 may be applicable to other medical devices and/or other therapies. In general, the techniques described in this disclosure may be implemented by any medical device, e.g., implantable or external, that utilizes an electrical lead within patient 14. As one alternative example, the techniques described herein may be implemented in implantable medical devices that generate electrograms for monitoring, but do not necessarily provide therapy to patient 14

In the example of FIG. 1, leads 18, 20, and 22 extend into the heart 12 of patient 14 to sense electrical activity of heart 12 and/or deliver electrical stimulation to heart 12. Leads 18, 20, and 22 may also be used to detect impedance values between any implanted electrodes within patient 14. In the example shown in FIG. 1, right ventricular (RV) lead 18 extends through one or more veins (not shown), the superior vena cava (not shown), and right atrium 26, and into right ventricle 28. Left ventricular (LV) coronary sinus lead 20 extends through one or more veins, the vena cava, right atrium 26, and into the coronary sinus 30 to a region adjacent to the free wall of left ventricle 32 of heart 12. Right atrial (RA) lead 22 extends through one or more veins and the vena cava, and into the right atrium 26 of heart 12.

In some examples, system 10 may additionally or alternatively include one or more leads or lead segments (not shown in FIG. 1) that deploy one or more electrodes within the vena cava, or other veins. Furthermore, in some examples, system 10 may additionally or alternatively include temporary or permanent epicardial or subcutaneous leads with electrodes implanted outside of heart 12, instead of or in addition to transvenous, intracardiac leads 18, 20 and 22. Such leads may be used for one or more of cardiac sensing, pacing, or cardioversion/defibrillation. For example, these electrodes may allow alternative electrical sensing configurations that provide improved or supplemental sensing in some patients. IMD 16 may use the techniques described herein to diagnose lead connection problems and lead fracture problems in any of these leads.

IMD 16 may sense electrical signals attendant to the depolarization and repolarization of heart 12 via electrodes (not shown in FIG. 1) coupled to at least one of the leads 18, 20, and 22. In some examples, IMD 16 provides pacing pulses to heart 12 based on the electrical signals sensed within heart 12. The configurations of electrodes used by IMD 16 for sensing and pacing may be unipolar or bipolar. IMD 16 may detect arrhythmia of heart 12, such as tachycardia or fibrillation of the atria 26 and 36 and/or ventricles 28 and 32, and may also provide defibrillation therapy and/or cardioversion therapy via electrodes located on at least one of the leads 18, 20, and 22. In some examples, IMD 16 may be programmed to deliver a progression of therapies, e.g., shocks with increasing energy levels, until a fibrillation of heart 12 is stopped. IMD 16 may detect fibrillation employing one or more fibrillation detection techniques known in the art.

In addition, IMD 16 may monitor the electrical signals of heart 12. IMD 16 may utilize any two or more electrodes carried on leads 18, 20, 22 to generate electrograms of cardiac activity. In some examples, IMD 16 may also use a housing electrode of IMD 16 (not shown) to generate electrograms and monitor cardiac activity. Although these electrograms may be used to monitor heart 12 for potential arrhythmias and other disorders for therapy, the electrograms may also be used to monitor the condition of heart 12. For example, IMD 16 may monitor heart rate, heart rate variability, ventricular heart rate, or other indicators of blood flow and the ability of heart 12 to pump blood.

During, or in addition to, monitoring the electrical signals of heart 12, IMD 16 may measure the impedance of one or more of leads 18, 20, and 22. An impedance measurement for a lead may be a measurement of an impedance of an electrical path that includes at least two electrodes, where at least one of the electrodes is located on the lead. A lead may include one or more electrodes, and there may be a variety of paths including one or more electrodes on the lead whose impedance may be considered an impedance for the lead. Such impedance measurements may be performed for each of leads 18, 20, and 22 numerous times after the leads are implanted within patient 14 to monitor the sensing integrity for each of the leads.

Periodic measurements of lead impedance may allow normal baseline impedances to be identified and variations in the lead impedance to be subsequently detected. Lead impedance tests, e.g. lead integrity checks, may be performed multiple times per day, once a day, one or more times per week, or any other frequency has determined by the clinician, manufacturer, or conditions of system 10 and/or patient 14. The impedance value, timing of any changes to the impedance value, and other characteristics may be analyzed to diagnose any problems with any of leads 18, 20, and 22. For example, impedance measurements may be used to diagnose and differentiate between lead connection problems and lead fracture problems.

IMD 16 may also analyze the electrical signal provided by leads, or the detection of cardiac events, e.g., ventricular depolarizations, within the electrical signal by the IMD, to monitor for oversensing of cardiac events within electrical signals provided by leads 18, 20, and 22. Noise may include any erratic signals with high frequency components, low frequency components, and/or a saturation of the signal. Noise caused by fractured leads, incomplete lead connections, or other hardware related conditions may be misinterpreted by IMD 16 as high frequency cardiac events. Distinguishing oversensing from high frequency cardiac events may be beneficial to avoid unnecessary intervention from IMD 16. Identifying oversensing may also be used to distinguish between lead fractures and lead connection problems. In some examples, the location of the lead fracture may also be detected. For example, a lead with a fracture inside the heart may result in oversensing synchronized to the cardiac cycle. Alternatively, a lead fracture outside the heart may result in oversensing asynchronized to the cardiac cycle.

When measuring impedance, oversensing, or any other characteristic of leads 18, 20, and 22, these types of analyses may be performed for each electrical circuit of system 10. In other words, each lead may include a separate electrical circuit for each electrode disposed on the lead. If each of leads 18, 20, and 22 has two separate electrodes, the impedance for each conductor electrically coupled to a respective electrode may be analyzed for integrity problems, e.g., the impedance of each conductor may be tested. Although a lead connection problem may create similar signals for each of the electrodes of that lead, a lead fracture may have occurred in only one of several conductors within the lead. For this reason, each distinct electrical circuit of leads 18, 20, and 22 may be tested regularly and analyzed for potential problems. IMD 16 may perform the integrity tests, e.g., impedance measurements, at regularly scheduled times, upon command from a user, upon identifying abnormal electrical sensing, e.g., oversensing, from a lead, and/or prior to delivering a therapy to patient 14.

Generally, the measured impedances of leads 18, 20, and 22 will be relatively low when there are no fractures within a lead and the connector pin of each lead is appropriately connected to header 34. These low impedance values may be within an average range, e.g., within a standard deviation of a baseline impedance value (an average of previous lead impedance measurements), or within a predetermined normal lead impedance range, for example. Although impedance values for leads 18, 20, and 22 may increase over time, e.g., due to changes in the electrode tissue interface, abrupt increases in lead impedance may indicate a lead connection or lead fracture problem. For example, very high impedance values may indicate a lead fracture problem. In another example, impedance values greater than the normal low impedance values may be associated with lead connection problems if the impedance returns to (or near) the low impedance baseline for a predetermined time or if the higher impedance value was detected within a certain time period from when the lead was connected to the IMD. In these examples, lead impedance measurements may be used to differentiate lead connection problems from lead fracture problems. This diagnosis may allow a clinician to reconnect a lead to the IMD when indicated instead of remove the lead from patient 14 when higher impedances are measured.

IMD 16 may also communicate with external programmer 24. In some examples, programmer 24 comprises a handheld computing device, computer workstation, or networked computing device. Programmer 24 may include a user interface that receives input from a user. In other examples, the user may also interact with programmer 24 remotely via a networked computing device. The user may interact with programmer 24 to communicate with IMD 16. For example, the user may interact with programmer 24 to retrieve physiological or diagnostic information from IMD 16. A user may also interact with programmer 24 to program IMD 16, e.g., select values for operational parameters of IMD 16. Although the user is a physician, technician, surgeon, electrophysiologist, or other healthcare professional, the user may be patient 14 in some examples.

For example, the user may use programmer 24 to diagnose any problems with lead integrity and/or lead connection problems with system 10. Although programmer 24 may retrieve this information, IMD 16 may instead push or transmit the lead integrity information to programmer 24 if one or more leads has a detected problem that may prevent appropriate therapy or result in delivery of unneeded shocks, for example, to heart 12. Although IMD 16 may diagnose problems with any of leads 18, 20, and 22 internally, IMD 16 may instead transmit collected lead impedance, oversensing, or other data to programmer 24 for processing and final diagnosis of lead fractures or lead connection problems. In other examples, programmer 24 may retrieve information from IMD 16 regarding the performance or integrity of IMD 16 or other components of system 10, in addition to leads 18, 20 and 22, such as a power source of IMD 16. In some examples, any of this information may be presented to the user as an alert (e.g., a notification or instruction). Further, alerts may be pushed from IMD 16 to facilitate alert delivery whenever programmer 24 or another computing device or computer network is detectable by IMD 16.

Programmer 24 may also allow the user to define how IMD 16 collects and/or analyzes any lead integrity data, e.g., timing of impedance measurements, thresholds for high impedance values, instructions for determining normal lead impedance values, instructions for diagnosing between lead connection and lead fracture problems, oversensing detection, or any other related information. For example, a clinician may use programmer 24 to instruct IMD 16 to measure and store one impedance measurement for each lead per day. In another example, programmer 24 may be used to instruct IMD 16 to analyze the previously collected and stored lead impedance values after each new measurement in order to diagnose any lead connection or lead fracture problems. In this manner, programmer 24 may be used to set or change any parameters of the lead integrity checks for diagnosis lead connection or lead fractures during use of system 10.

IMD 16 and programmer 24 may communicate via wireless communication using any techniques known in the art. Examples of communication techniques may include, for example, low frequency or radiofrequency (RF) telemetry, but other techniques are also contemplated. In some examples, programmer 24 may include a programming head that may be placed proximate to the patient's body near the IMD 16 implant site in order to improve the quality or security of communication between IMD 16 and programmer 24.

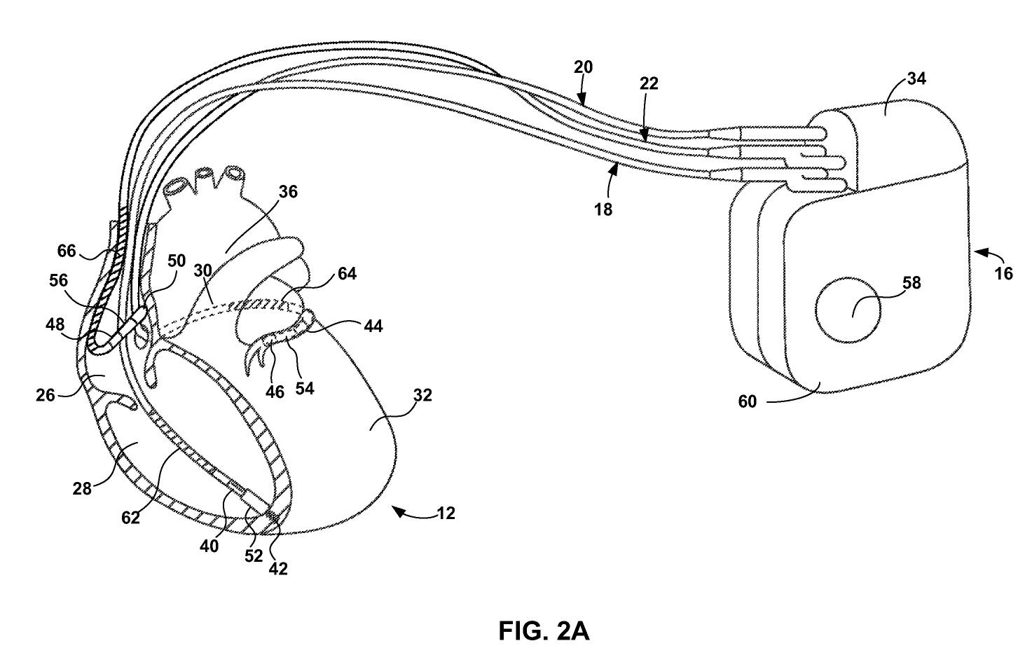

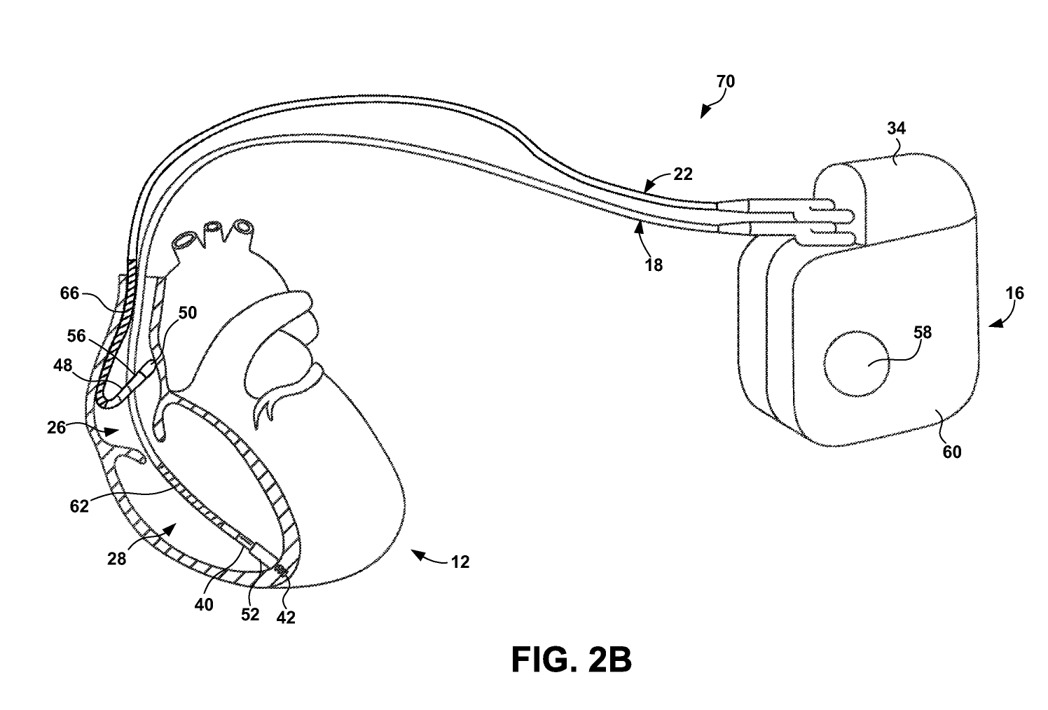

FIG. 2A is a conceptual drawing illustrating example IMD 16 and leads 18, 20, and 22 of system 10 in greater detail. As shown in FIG. 2A, IMD 16 is coupled to leads 18, 20, and 22. Leads 18, 20, and 22 may be electrically coupled to a signal generator, e.g., stimulation generator, and a sensing module of IMD 16 via connector block 34. In some examples, proximal ends of leads 18, 20, and 22 may include electrical contacts that electrically couple to respective electrical contacts within connector block 34 of IMD 16. In addition, in some examples, leads 18, 20, and 22 may be mechanically coupled to connector block 34 with the aid of set screws, connection pins, snap connectors, or another suitable mechanical coupling mechanism.

Each of the leads 18, 20, and 22 includes an elongated insulative lead body, which may carry a number of concentric coiled conductors separated from one another by tubular insulative sheaths. Bipolar electrodes 40 and 42 are located adjacent to a distal end of lead 18 in right ventricle 28. In addition, bipolar electrodes 44 and 46 are located adjacent to a distal end of lead 20 in coronary sinus 30 and bipolar electrodes 48 and 50 are located adjacent to a distal end of lead 22 in right atrium 26. In the illustrated example, there are no electrodes located in left atrium 36. However, other examples may include electrodes in left atrium 36.

Electrodes 40, 44 and 48 may take the form of ring electrodes, and electrodes 42, 46 and 50 may take the form of extendable helix tip electrodes mounted retractably within insulative electrode heads 52, 54 and 56, respectively. In other examples, one or more of electrodes 42, 46 and 50 may take the form of small circular electrodes at the tip of a tined lead or other fixation element. Leads 18, 20, and 22 also include elongated electrodes 62, 64, 66, respectively, which may take the form of a coil. Each of the electrodes 40, 42, 44, 46, 48, 50, 62, 64 and 66 may be electrically coupled to a respective one of the coiled conductors within the lead body of its associated lead 18, 20, and 22, and thereby coupled to respective ones of the electrical contacts on the proximal end of leads 18, 20 and 22.

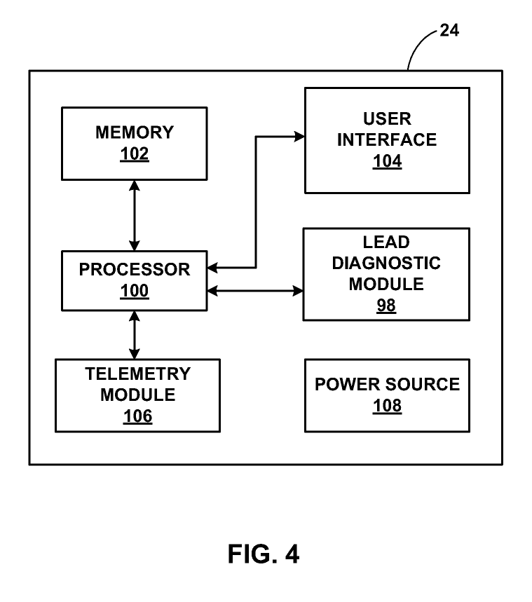

In some examples, as illustrated in FIG. 2A, IMD 16 includes one or more housing electrodes, such as housing electrode 58, which may be formed integrally with an outer surface of hermetically-sealed housing 60 of IMD 16 or otherwise coupled to housing 60. In some examples, housing electrode 58 is defined by an uninsulated portion of an outward facing portion of housing 60 of IMD 16. Other division between insulated and uninsulated portions of housing 60 may be employed to define two or more housing electrodes. In some examples, housing electrode 58 comprises substantially all of housing 60. As described in further detail with reference to FIG. 4, housing 60 may enclose a signal generator that generates therapeutic stimulation, such as cardiac pacing pulses and defibrillation shocks, as well as a sensing module for monitoring the rhythm of heart 12.

IMD 16 may sense electrical signals attendant to the depolarization and repolarization of heart 12 via electrodes 40, 42, 44, 46, 48, 50, 62, 64 and 66. The electrical signals are conducted to IMD 16 from the electrodes via the respective leads 18, 20, 22. IMD 16 may sense such electrical signals via any bipolar combination of electrodes 40, 42, 44, 46, 48, 50, 62, 64 and 66. Furthermore, any of the electrodes 40, 42, 44, 46, 48, 50, 62, 64 and 66 may be used for unipolar sensing in combination with housing electrode 58. The combination of electrodes used for sensing may be referred to as a sensing configuration or electrode vector.

In some examples, IMD 16 delivers pacing pulses via bipolar combinations of electrodes 40, 42, 44, 46, 48 and 50 to produce depolarization of cardiac tissue of heart 12. In some examples, IMD 16 delivers pacing pulses via any of electrodes 40, 42, 44, 46, 48 and 50 in combination with housing electrode 58 in a unipolar configuration. Furthermore, IMD 16 may deliver defibrillation pulses to heart 12 via any combination of elongated electrodes 62, 64, 66, and housing electrode 58. Electrodes 58, 62, 64, 66 may also be used to deliver cardioversion pulses to heart 12. Electrodes 62, 64, 66 may be fabricated from any suitable electrically conductive material, such as, but not limited to, platinum, platinum alloy or other materials known to be usable in implantable defibrillation electrodes. The combination of electrodes used for delivery of stimulation or sensing, their associated conductors and connectors, and any tissue or fluid between the electrodes, may define an electrical path.

The configuration of system 10 illustrated in FIGS. 1 and 2A is merely one example. In other examples, a system may include epicardial leads and/or subcutaneous electrodes instead of or in addition to the transvenous leads 18, 20, 22 illustrated in FIG. 1. Further, IMD 16 need not be implanted within patient 14. In examples in which IMD 16 is not implanted in patient 14, IMD 16 may sense electrical signals and/or deliver defibrillation pulses and other therapies to heart 12 via percutaneous leads that extend through the skin of patient 14 to a variety of positions within or outside of heart 12. Further, external electrodes or other sensors may be used by IMD 16 to deliver therapy to patient 14 and/or sense and detect patient metrics used to generate a heart failure risk score.

In addition, in other examples, a system may include any suitable number of leads coupled to IMD 16, and each of the leads may extend to any location within or proximate to heart 12. For example, other examples of systems may include three transvenous leads located as illustrated in FIGS. 1 and 2, and an additional lead located within or proximate to left atrium 36. As another example, other examples of systems may include a single lead that extends from IMD 16 into right atrium 26 or right ventricle 28, or two leads that extend into a respective one of the right ventricle 26 and right atrium 26. An example of a two lead type of system is shown in FIG. 2B. Any electrodes located on these additional leads may be used in sensing and/or stimulation configurations.

Lead connection problems or lead fracture problems may be diagnosed with regard to any of leads 18, 20, 22, or any other leads electrically coupled to IMD 16. In addition, IMD 16 may even diagnose lead connection problems or lead fractures with other leads coupled to different implantable devices. IMD 16 may communicate with the other implantable medical device to request impedance measurements, receive impedance measurements, analyze impedance measurements and any oversensing, or any other tasks IMD 16 may perform with regard to coupled leads 18, 20, and 22 in the manner described herein.

FIG. 2B is a conceptual drawing illustrating another example system 70, which is similar to system 10 of FIGS. 1 and 2, but includes two leads 18 and 22, rather than three leads. Leads 18 and 22 are implanted within right ventricle 28 and right atrium 26, respectively. System 70 shown in FIG. 2B may be useful for physiological sensing and/or providing pacing, cardioversion, or other therapies to heart 12. Diagnosing lead connection problems or lead fracture problems according to this disclosure may be performed in two lead systems in the manner described herein with respect to three lead systems. In other examples, a system similar to systems 10 and 70 may only include one lead (e.g., any of leads 18, 20 or 22) to deliver therapy and/or sense patient conditions.

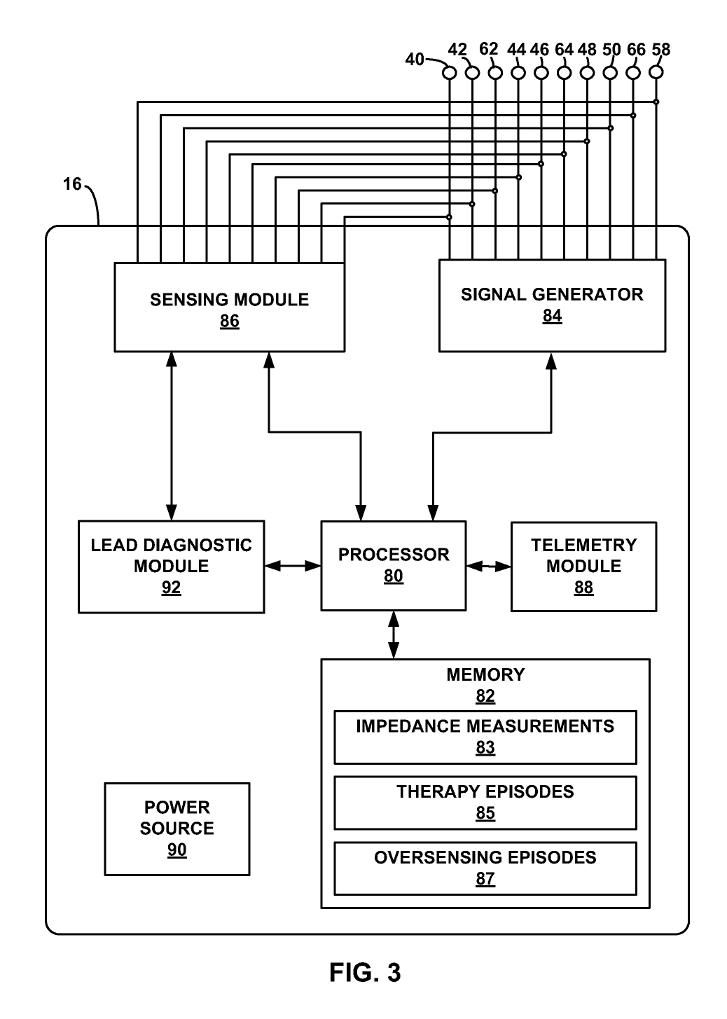

FIG. 3 is a functional block diagram illustrating an example configuration of IMD 16 of FIG. 1. In the illustrated example, IMD 16 includes a processor 80, memory 82, lead diagnostic module 92, signal generator 84, sensing module 86, telemetry module 88, and power source 90. Memory 82 includes computer-readable instructions that, when executed by processor 80, cause IMD 16 and processor 80 to perform various functions attributed to IMD 16 and processor 80 herein. Memory 82 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media.

Processor 80 may include any one or more of a microprocessor, a controller, a digital signal processor (DSP), an application specific integrated circuit (ASIC), a field-programmable gate array (FPGA), or equivalent discrete or analog logic circuitry. In some examples, processor 80 may include multiple components, such as any combination of one or more microprocessors, one or more controllers, one or more DSPs, one or more ASICs, or one or more FPGAs, as well as other discrete or integrated logic circuitry. The functions attributed to processor 80 herein may be embodied as software, firmware, hardware or any combination thereof.

Processor 80 controls signal generator 84 to deliver stimulation therapy to heart 12 according to selected values for operational parameters, which may be stored in memory 82. For example, processor 80 may control stimulation generator 84 to deliver electrical pulses with the amplitudes, pulse widths, frequency, or electrode polarities specified by the operational parameter values, and at times relative to detection or non-detection of cardiac events as specified by the operational parameter values.

Signal generator 84 is electrically coupled to electrodes 40, 42, 44, 46, 48, 50, 58, 62, 64, and 66, e.g., via conductors of the respective lead 18, 20, and 22, or, in the case of housing electrode 58, via an electrical conductor disposed within housing 60 of IMD 16. In the illustrated example, signal generator 84 is configured to generate and deliver electrical stimulation therapy to heart 12. For example, signal generator 84 may deliver defibrillation shocks to heart 12 via at least two electrodes 58, 62, 64, 66. Signal generator 84 may deliver pacing pulses via ring electrodes 40, 44, 48 coupled to leads 18, 20, and 22, respectively, and/or helical electrodes 42, 46, and 50 of leads 18, 20, and 22, respectively. In some examples, signal generator 84 delivers pacing, cardioversion, or defibrillation stimulation in the form of electrical pulses. In other examples, signal generator may deliver one or more of these types of stimulation in the form of other signals, such as sine waves, square waves, or other substantially continuous time signals.

Signal generator 84 may include a switch module and processor 80 may use the switch module to select, e.g., via a data/address bus, which of the available electrodes are used to deliver pacing, cardioversion, or defibrillation stimulation. The switch module may include a switch array, switch matrix, multiplexer, or any other type of switching device suitable to selectively couple stimulation energy to selected electrodes.

Electrical sensing module 86 monitors signals from at least one of electrodes 40, 42, 44, 46, 48, 50, 58, 62, 64 or 66 in order to monitor electrical activity of heart 12. Sensing may be done to detect cardiac events, e.g., depolarizations, and thereby determine heart rates and detect arrhythmias. Sensing module 86 may also include a switch module to select which of the available electrodes are used to sense the heart activity, depending upon which electrode combination, or electrode vector, is used in the current sensing configuration. In some examples, processor 80 may select the electrodes that function as sense electrodes, i.e., select the sensing configuration, via the switch module within sensing module 86. Sensing module 86 may include one or more detection channels, each of which may be coupled to a selected electrode configuration for detection of cardiac signals via that electrode configuration. Some detection channels may be configured to detect particular cardiac events, such as P-waves or R-waves, and provide indications of the occurrences of such events to processor 80, e.g., as described in U.S. Pat. No. 5,117,824 to Keimel et al., which issued on Jun. 2, 1992 and is entitled, “APPARATUS FOR MONITORING ELECTRICAL PHYSIOLOGIC SIGNALS,” and is incorporated herein by reference in its entirety. A sensed P-wave indicates an atrial depolarization, while a sensed R-wave indicates a ventricular depolarization. Processor 80 may control the functionality of sensing module 86 by providing signals via a data/address bus.

Processor 80 may include a timing and control module, which may be embodied as hardware, firmware, software, or any combination thereof. The timing and control module may comprise a dedicated hardware circuit, such as an ASIC, separate from other processor 80 components, such as a microprocessor, or a software module executed by a component of processor 80, which may be a microprocessor or ASIC. The timing and control module may implement programmable counters. If IMD 16 is configured to generate and deliver pacing pulses to heart 12, such counters may control the basic time intervals associated with DDD, VVI, DVI, VDD, AAI, DDI, DDDR, VVIR, DVIR, VDDR, AAIR, DDIR and other modes of pacing.

Intervals defined by the timing and control module within processor 80 may include atrial and ventricular pacing escape intervals, refractory periods during which sensed P-waves and R-waves are ineffective to restart timing of the escape intervals, and the pulse widths of the pacing pulses. As another example, the timing and control module may withhold sensing from one or more channels of sensing module 86 for a time interval during and after delivery of electrical stimulation to heart 12. The durations of these intervals may be determined by processor 80 in response to stored data in memory 82. The timing and control module of processor 80 may also determine the amplitude of the cardiac pacing pulses.

Interval counters implemented by the timing and control module of processor 80 may be reset upon sensing of R-waves and P-waves with detection channels of sensing module 86. In examples in which IMD 16 provides pacing, signal generator 84 may include pacer output circuits that are coupled, e.g., selectively by a switching module, to any combination of electrodes 40, 42, 44, 46, 48, 50, 58, 62, or 66 appropriate for delivery of a bipolar or unipolar pacing pulse to one of the chambers of heart 12. In such examples, processor 80 may reset the interval counters upon the generation of pacing pulses by signal generator 84, and thereby control the basic timing of cardiac pacing functions, including anti-tachyarrhythmia pacing.

The value of the count present in the interval counters when reset by sensed R-waves and P-waves may be used by processor 80 to measure the durations of R-R intervals, P-P intervals, P-R intervals and R-P intervals, which are measurements that may be stored in memory 82. Processor 80 may use the count in the interval counters to detect a tachyarrhythmia event, such as VF or VT. These intervals may also be used to detect the overall heart rate, ventricular contraction rate, and heart rate variability. A portion of memory 82 may be configured as a plurality of recirculating buffers, capable of holding series of measured intervals, which may be analyzed by processor 80 in response to the occurrence of a pace or sense interrupt to determine whether the patient's heart 12 is presently exhibiting atrial or ventricular tachyarrhythmia.

In some examples, an arrhythmia detection method may include any suitable tachyarrhythmia detection algorithms. In one example, processor 80 may utilize all or a subset of the rule-based detection methods described in U.S. Pat. No. 5,545,186 to Olson et al., entitled, “PRIORITIZED RULE BASED METHOD AND APPARATUS FOR DIAGNOSIS AND TREATMENT OF ARRHYTHMIAS,” which issued on Aug. 13, 1996, or in U.S. Pat. No. 5,755,736 to Gillberg et al., entitled, “PRIORITIZED RULE BASED METHOD AND APPARATUS FOR DIAGNOSIS AND TREATMENT OF ARRHYTHMIAS,” which issued on May 26, 1998. U.S. Pat. No. 5,545,186 to Olson et al. U.S. Pat. No. 5,755,736 to Gillberg et al. is incorporated herein by reference in their entireties. However, other arrhythmia detection methodologies may also be employed by processor 80 in other examples.

In some examples, processor 80 may determine that tachyarrhythmia has occurred by identification of shortened R-R (or P-P) interval lengths. Generally, processor 80 detects tachycardia when the interval length falls below 220 milliseconds (ms) and fibrillation when the interval length falls below 180 ms. These interval lengths are merely examples, and a user may define the interval lengths as desired, which may then be stored within memory 82. This interval length may need to be detected for a certain number of consecutive cycles, for a certain percentage of cycles within a running window, or a running average for a certain number of cardiac cycles, as examples.

In the event that processor 80 detects an atrial or ventricular tachyarrhythmia based on signals from sensing module 86, and an anti-tachyarrhythmia pacing regimen is desired, timing intervals for controlling the generation of anti-tachyarrhythmia pacing therapies by signal generator 84 may be loaded by processor 80 into the timing and control module to control the operation of the escape interval counters therein and to define refractory periods during which detection of R-waves and P-waves is ineffective to restart the escape interval counters for the an anti-tachyarrhythmia pacing. In the event that processor 80 detects an atrial or ventricular tachyarrhythmia based on signals from sensing module 86, and a cardioversion or defibrillation shock is desired, processor 80 may control the amplitude, form and timing of the shock delivered by signal generator 84.

If there are any lead fracture problems or lead connection problems with leads 18, 20, or 22, IMD 16 may not be able to properly detect intrinsic cardiac events necessary to identify when intervention therapy is necessary or detect why type of therapy needs to be delivered to patient 14. Therefore, diagnosing lead fractures and lead connection problems may allow a clinician and patient to minimize improper operation by IMD 16.

To facilitate diagnosis of lead fractures and lead connection problems, processor 80 may control the performance of impedance measurements by signal generator 84 and sensing module 86. The impedance measured may be of any of a variety of electrical paths that include two or more of electrodes 40, 42, 44, 46, 48, 50, 58, 62, 64 and 66. In particular, sensing module 86 may include circuitry to measure an electrical parameter value during delivery of an electrical signal between at least two of the electrodes by signal generator 84.

Processor 80 may control signal generator 84 to deliver the electrical signal between the electrodes. Processor 80 may determine impedance values based on parameter values measured by sensing module 86. In some examples, processor 80 may perform an impedance measurement by controlling delivery, from signal generator 84, of a voltage pulse between first and second electrodes. Sensing module 86 may measure a resulting current, and processor 80 may calculate a resistance based upon the voltage amplitude of the pulse and the measured amplitude of the resulting current. In other examples, processor 80 may perform an impedance measurement by controlling delivery, from signal generator 84, of a current pulse between first and second electrodes. Sensing module 86 may measure a resulting voltage, and processor 80 may calculate a resistance based upon the current amplitude of the pulse and the measured amplitude of the resulting voltage. Sensing module 86 may include circuitry for measuring amplitudes of resulting currents or voltages, such as sample and hold circuitry, as well as analog to digital converter circuitry for providing a digital value representing the measured voltage or current amplitude to processor and/or lead diagnostic module 92.

In these examples of performing impedance measurements, signal generator 84 delivers signals that do not necessarily deliver stimulation therapy to heart 12, due to, for example, the amplitudes of such signals and/or the timing of delivery of such signals. For example, these signals may comprise sub-threshold amplitude signals that may not stimulate heart 12. In some cases, these signals may be delivered during a refractory period, in which case they also may not stimulate heart 12. IMD 16 may use defined or predetermined pulse amplitudes, widths, frequencies, or electrode polarities for the pulses delivered for these various impedance measurements. In some examples, the amplitudes and/or widths of the pulses may be sub-threshold, e.g., below a threshold necessary to capture or otherwise activate tissue, such as cardiac tissue.

In certain cases, IMD 16 may collect impedance values that include both a resistive and a reactive (i.e., phase) component. In such cases, IMD 16 may measure impedance during delivery of a sinusoidal or other time varying signal by signal generator 84, for example. Thus, as used herein, the term “impedance” is used in a broad sense to indicate any collected, measured, and/or calculated value that may include one or both of resistive and reactive components. Impedance data may include actual, measured impedance values, or may include values that can be used to calculate impedance (such as current and/or voltage values).

Memory 82 may be configured to store a variety of operational parameters, sensed and detected data, and any other information related to the therapy and treatment of patient 14. In the example of FIG. 3, memory 82 also includes impedance measurements 83, therapy episodes 85, and oversensing episodes 87. Impedance measurements 83 may include some or all of the impedance values measured for each of the electrical paths provided by leads 18, 20, and 22, which may be used by lead diagnostic module 92 to diagnose lead connection problems and lead fractures. Impedance measurements 83 may include individual previously measured impedance values, averages of measured impedance values, and/or impedance profiles over time for each lead. Impedance measurements 83 may include historical impedance measurements for each lead 18, 20, and 22, e.g., any impedance measurements taken since the lead was implanted and/or connected to IMD 16. In other examples, impedance measurements 83 may only store those impedance measurements required by lead diagnostic module 92 to diagnose lead connection and lead fracture problems.

Therapy episodes 85 may store information regarding any sensed episodes of cardiac activity for which a responsive therapy was delivered to patient 14 by IMD 16. For example, therapy episodes 85 may include information regarding any episodes for which shocks and/or pacing delivered to patient 14, as well as information regarding pacing therapy generally, e.g., percent pacing. Therapy episodes 85 may also include those events which called for therapy and therapy was not delivered due to one or more inconsistencies in the detected episodes or problems detected with patient 14 or components of system 10, e.g., leads 18, 20, or 22. Additionally, therapy episodes 85 may store the parameters and/or programs of any therapy delivered in response to the episode being detected. Both impedance measurements 83 and therapy episodes 85 may store time and date information for each impedance measurement and therapy episode, respectively. Therapy episodes 85 may be used by lead diagnostic module 92 or other device to determine if oversensing is occurring with the lead.

Although all of stored therapy episodes 87 may be used when diagnosing an impedance issue with a lead, in other examples lead diagnostic module 92 may only utilize a subset of the stored therapy episodes 87 when diagnosing a lead fracture or lead connection problem. The subset may include episodes that are relatively proximate to e.g., occurred just prior to or after, such as within a week of, the detection of an increased impedance value. Therapy episodes that have occurred more than a week prior to the increased impedance value, for example, may have occurred when the lead was functioning appropriately. However, therapy episodes that occurred just prior to an increase in impedance, e.g., within a day or a week, and therapy episodes detected after the increased impedance value may be used to determine if any oversensing is occurring with the lead.

Oversensing episodes 87 may store information related to any possible oversensing events detected by processor 80. For example, oversensing episodes 87 may include non-sustained tachyarrhythmia episodes, e.g., more than four tachyarrhythmia beats but less than twelve tachyarrhythmia beats, and/or a count of short intervals, e.g., intervals detected to be too short to be physiological heart beat intervals. In other examples, oversensing episodes 87 may include morphologies of cardiac signals associated with non-sustained tachyarrhythmias or short intervals. In this manner, lead diagnostic module 92 may use oversensing episodes 87 to determine whether a lead fracture or lead connection problem is present. Similar to therapy episodes 85, only those oversensing episodes 87 that have occurred proximate to a detected increase in lead impedance may be used to diagnose a lead fracture or lead connection problem.

In some examples, memory 82 may also store instructions for diagnosing lead connection problems and lead fracture problems. These instructions may include when to perform the diagnosis, thresholds for impedance values (e.g., abrupt rise thresholds, high impedance values, and normal impedance values) and oversensing, time thresholds between measured impedance values, and/or when to incorporate therapy episodes 85 or oversensing information from oversensing episodes 87 into the diagnosis. Lead diagnostic module 92 may utilize this information stored in memory 82, or in other examples, lead diagnostic module 92 may itself store diagnosis instructions.

Lead diagnostic module 92 may perform some or all of the diagnosis of lead connection problems or lead fracture problems. This diagnosis, may in some examples, include a differentiation between a lead fracture and a lead connection problem based upon measured impedance values, the timing of impedance values, the timing of an increased impedance value in relation to an event, any oversensing, and/or the presence of therapy episodes 85. It is noted that functions attributed to lead diagnostic module 92 herein may be embodied as software, firmware, hardware or any combination thereof. In some examples, lead diagnostic module 92 may at least partially be implemented in, e.g., a software process executed by, processor 80.

In one example, lead diagnostic module 92 may determine a timing of an increased impedance value from a plurality of impedance values associated with one of leads 18, 20, and 22 and stored in impedance measurements 83. The increased impedance value may be greater than an impedance threshold that is set above a baseline impedance value, e.g., an average of previous impedance measurements. The baseline impedance value may be a running average, weighted average, or recent average that represents the normal impedance values of the lead. The normal impedance values may be those impedance values associated with normal operating conditions that include no lead fractures and a complete connection between the pin of the lead and the header of IMD 16. Based on the timing of the increased impedance value, lead diagnostic module 92 may select between a diagnosis of a lead fracture or a diagnosis of a lead connection problem.

In some examples, lead diagnostic module 92 may determine whether the increased impedance value occurred within an interval having a predetermined duration. In some examples, the increased impedance may be a second or subsequent episode of increased impedance, and the interval may begin at a point at which the measured impedance values returned to a baseline or average value, or to a value near the baseline or average value, after a previous episode of increased impedance. In other words, the duration of a period in which measured impedance values are at or near a baseline impedance value after having been at increased values may be relevant for diagnosing a lead fracture or lead connection problem. In one example, the interval of time during which the impedance must have returned to and remained near the baseline to be considered a return to baseline, e.g., the predetermined duration threshold for a return to baseline event, may be approximately 45 days. In some examples, the duration threshold may be generally between approximately 15 days and 90 days.

In some examples, the interval may begin at the time that leads 18, 20, or 22 were connected to IMD 16. The duration of a period between when the lead was connected to IMD 16 and when the increased impedance value was measured may be used to differentiate between a lead fracture and an incomplete lead connection to IMD 16. For example, lead diagnostic module 92 may automatically diagnose a lead connection problem when the increased impedance value occurred less than a duration threshold of approximately 200 days from the connection of IMD 16 to the respective one of leads 18, 20, or 22. In some examples, the duration threshold from the connection of the lead to IMD 16 within which occurrence of an increased impedance value will lead to diagnosis of a connection problem may be generally between approximately 100 days and 2 years. As described above, the duration intervals may be predetermined intervals. However, the duration intervals may be dependent upon other events or patient conditions in some examples.

In some examples, lead diagnostic module 92 may diagnose a lead connection problem immediately if the increased impedance value occurs within a time interval having a predetermined duration threshold of connecting the one of leads 18, 20, or 22 to IMD 16. This diagnosis may be made regardless of any other detected events or impedances because of the impedance increase so soon after connection between IMD 16 and any one of leads 18, 20, or 22 may be highly indicative of a problem with the connection of the lead to the IMD instead of a lead fracture. The predetermined duration threshold in this case may be approximately 30 days from connection of the lead, in one example. In other examples, the predetermined duration threshold may be generally between 10 days and 90 days.

In some examples, the increased impedance value detected by lead diagnostic module 92 may be considered an abrupt rise in the impedance magnitude, e.g., a change of sufficient magnitude relative to the baseline impedance value (e.g., the average of previous impedance measurements) within a sufficiently short period of time to be classified as abrupt. An abrupt rise in impedance magnitude may indicate a structural change in leads 18, 20, or 22 instead of a change with the physiological or anatomical environment in which the impedance measurement was taken. For example, an abrupt rise in impedance magnitude may be a single impedance measurement of at least approximately 350 ohms relative to the baseline impedance value, or approximately 60 percent greater than the baseline impedance value. In other examples, the abrupt rise threshold for an increased impedance value may be generally between approximately 100 and 1000 ohms greater than the baseline impedance value or between approximately 20 percent and 200 percent greater than the baseline impedance value.

Although the detection of a single impedance measurement above a threshold may be used to detect an abrupt rise in impedance magnitude, other examples may require two or more impedance measurements above the threshold before an abrupt rise is determined. To result in identification of an abrupt rise in impedance, these multiple impedance measurements above the threshold may be required to be consecutive, or to have occurred within a predetermined time period, e.g., X of Y impedance measurements above the threshold. In addition, the frequency of impedance measurements may increase (e.g., increase from once a day to once an hour or once an hour to once a minute) upon detection of the first increased impedance measurement above the threshold. This increased frequency of impedance of measurements may be used to more expediently determine or confirm the presence of an abrupt rise in impedance magnitude. The increased frequency of impedance measurements may continue for a predetermined duration, e.g., 24 hours or 1 week, or until the measured impedance is classified as above a maximum impedance threshold or classified as below an increased impedance threshold, e.g., classified as having returned to baseline impedance. In some examples, increasing the frequency of impedance measurements may not increase the frequency for updating the baseline impedance value, or alternatively, the baseline impedance value may not be updated at all until the increased impedance measurement frequency ceases.

The baseline impedance value may generally be the operational impedance value, or range, of leads 18, 20, and 22 when there are no lead fractures, incomplete lead connections, or any other problems. This baseline may be a rolling average, a weighted average, a long term average, or another measure or combination of measures of previously determined impedance values indicative of normal operational lead impedances.

Lead diagnostic module 92 may also determine a maximum impedance value from the measured impedance values, and lead diagnostic module 92 may diagnose a lead fracture when the measured impedance value is greater than a maximum impedance threshold. In one example, the maximum impedance threshold may be approximately 10,000 ohms. In other examples, the maximum impedance threshold may be set between approximately 4,000 ohms and 15,000 ohms. Other thresholds outside of this range are contemplated as well, depending on the configuration of leads 18, 20, and 22 and IMD 16.

In addition, lead diagnostic module 92 may diagnose problems with leads 18, 20, or 22 based on detecting stable high impedance values and oversensing. Lead diagnostic module 92 may determine the occurrence of a stable high impedance level based impedance measurements 83. Detection of a stable high impedance level may include detecting consecutive impedance values greater than a stable high impedance magnitude threshold. The detection of the stable high impedance level may occur after first identifying an abrupt rise in the impedance values.

The stable high impedance magnitude threshold may be determined as a percentage or fraction of the maximum measured impedance value. The maximum measured impedance value may be the impedance value or values identified as the abrupt rise in impedance or a greater impedance value following the abrupt rise. In one example, the stable high impedance magnitude threshold may be set to 65 percent of the maximum measured impedance value. In this example, a stable high impedance level may be determined or identified if the minimum measured impedance value over a period of time subsequent to the abrupt rise in impedance values is equal to or greater than 65 percent of the maximum measured impedance value subsequent to the abrupt rise in impedance values. In other examples, the stable high impedance magnitude threshold may be between approximately 30 and 90 percent of the maximum measured impedance value.

In alternative examples, a stable high impedance magnitude threshold may not be based on the maximum measured impedance value. Instead, a stable high impedance level may be determined when a plurality of impedance values remain above any threshold. The stable high impedance magnitude threshold may be based on the baseline impedance value, e.g., a certain percentage or magnitude above the baseline impedance value. For example, the stable high impedance magnitude may be set as low as the increased impedance threshold used to detect an abrupt rise in impedance. In other examples, the stable high impedance threshold may be based on a percentage of the increased impedance value or values identified as the abrupt rise in impedance.

Detection of a stable high impedance level may also require detection of a threshold number of consecutive impedance values exceeding the stable high impedance magnitude threshold, or that all impedance values over a certain period of time exceed the stable high impedance magnitude threshold. In one example, a stable high impedance level is only determined if the measured impedance values remain above the stable high impedance threshold for at least two weeks after detection of an increased impedance value (e.g., an abrupt rise in impedance). In other examples, a stable high impedance level may require between 5 and 20 consecutive impedance values or consecutive impedance values for between 7 days and 30 days that exceed the stable high impedance magnitude threshold. However, stable high impedance levels may be defined with shorter or longer periods of times. Alternatively, it may not be required that consecutive measured impedance values be above a stable high impedance magnitude threshold to classify the measured impedances as being indicative of a stable high impedance. For example, a predetermined number of impedance values, a predetermined frequency of values, or a supermaj ority of impedance values above the stable high impedance magnitude threshold may be sufficient to detect a stable high impedance level. Generally, the determination of a stable high impedance level occurs after the detection of an abrupt rise in impedance.

Lead diagnostic module 92 may also determine whether oversensing occurred in the cardiac event sensing by IMD 16 based on the signals from one of leads 18, 20, or 22. The oversensing may be detected when cardiac events are being detected more frequently than actual cardiac events occur because noise is interfering with correct sensing of intrinsic cardiac signals. Lead diagnostic module 92 may diagnose a lead fracture if both a stable high impedance level and oversensing is determined from the impedance measurements. If a stable high impedance level is determined with no oversensing, lead diagnostic module 92 may still diagnose the lead as functioning properly, in some examples. As described herein, oversensing events 87 may include information used by lead diagnostic module 92 to determine if any oversensing has occurred.

As described herein, sensing module 86 may be used to measure each of the impedance values stored in memory 82 as impedance measurements 83. However, lead diagnostic module 92 may calibrate, modify, or otherwise process the measured impedance values prior to the measurements being stored as impedance measurements 83. Processor 80 may generally store impedance measurements 83 in memory 82, but lead diagnostic module 92 may store the impedance values in other examples. Lead diagnostic module 92 may generate diagnoses of lead connection or lead fracture problems with impedance measurements 83 and one or more new impedance measurement not yet stored in memory 82. However, in other examples lead diagnostic module 92 may only analyze impedance measurements 83 stored in memory 82 before generating a diagnosis.

In some examples, IMD 16 may additionally utilize an activity sensor (not shown) that may include one or more accelerometers or other devices capable of detecting motion and/or position of patient 14. The activity sensor may therefore detect activities of patient 14 or postures engaged by patient 14. The detected activities may, in some examples, be used to detect episodes of patient 14 and/or monitor patient 14 response to therapy. In other examples, the diagnosis of lead connection problems or lead fractures may include the use of patient activity information as part of the analysis.

In some examples, processor 80 may provide an alert to a user, e.g., of programmer 24, regarding the diagnosis of a lead connection problem or a lead fracture. In one example, processor 80 may provide an alert with the diagnosis when programmer 24 or another device communicates with IMD 16. In other examples, processor 80 may push an alert to programmer 24 or another device whenever the diagnosis of a lead connection problem or lead fracture indicates patient 14 is a risk of a potentially harmful therapy or absence of needed therapy due to the diagnosed problem. Alternatively, IMD 16 may directly indicate to patient 14 that leads 18, 20, or 22 need maintenance from a clinician. IMD 16 may include a speaker to emit an audible sound through the skin of patient 14 or a vibration module that vibrates to notify patient 14 of needed medical attention. Processor 80 may choose this action, for example, if the alert cannot be sent because of no available connection.

Telemetry module 88 includes any suitable hardware, firmware, software or any combination thereof for communicating with another device, such as programmer 24 (FIG. 1). Under the control of processor 80, telemetry module 88 may receive downlink telemetry from and send uplink telemetry to programmer 24 with the aid of an antenna, which may be internal and/or external. Processor 80 may provide the data to be uplinked to programmer 24 and the control signals for the telemetry circuit within telemetry module 88, e.g., via an address/data bus. In some examples, telemetry module 88 may provide received data to processor 80 via a multiplexer. The data sent by telemetry module 88 may be the diagnosis or lead integrity data required for an external device to generate the diagnosis.

Using telemetry module 88, IMD 16 may present the automatically selected diagnosis from lead diagnostic module 92 to a user. Telemetry module 88 may communicate directly with an external device that presents the diagnosis to a user. In this manner, the diagnosis may prevent unnecessary explantation of the medical lead when the diagnosis is the lead connection problem. In other words, increases in lead impedance, for example, would not always be treated as a lead fracture that requires replacement.

In some examples, processor 80 may transmit atrial and ventricular heart signals, e.g., EGMs, produced by atrial and ventricular sense amplifier circuits within sensing module 86 to programmer 24. Programmer 24 may interrogate IMD 16 to receive the heart signals. Processor 80 may store heart signals within memory 82, and retrieve stored heart signals from memory 82. Processor 80 may also generate and store marker codes indicative of different cardiac events that sensing module 86 detects, and transmit the marker codes to programmer 24. An example pacemaker with marker-channel capability is described in U.S. Pat. No. 4,374,382 to Markowitz, entitled, “MARKER CHANNEL TELEMETRY SYSTEM FOR A MEDICAL DEVICE,” which issued on Feb. 15, 1983 and is incorporated herein by reference in its entirety.

In some examples, IMD 16 may signal programmer 24 to further communicate with and pass the alert or other form of the lead integrity diagnosis through a network such as the Medtronic CareLink® Network developed by Medtronic, Inc., of Minneapolis, Minn., or some other network linking patient 14 to a clinician. In this manner, a computing device or user interface of the network may be the external computing device that delivers the alert, e.g., the diagnosis of a lead connection problem or a lead fracture, to the user.

The various components of IMD 16 are coupled to power source 90, which may include a rechargeable or non-rechargeable battery. A non-rechargeable battery may be capable of holding a charge for several years, while a rechargeable battery may be inductively charged from an external device, e.g., on a daily or weekly basis. In other examples, power source 90 may include a supercapacitor.

In alternative examples, processor 80 may utilize the diagnosis to alter sensing of cardiac events and/or deliver of therapy to patient 14. If a lead is diagnosed with a lead connection problem or a lead fracture, processor 80 may remove any electrical circuits utilizing the affected lead from monitoring or therapy. Processor 80 may also switch to alternative operational electrodes and/or leads to maintain cardiac event monitoring and/or therapy delivery. Therefore, IMD 16 may be able to automatically adjust therapy from the diagnosis to still treat patient 14 until a problem lead can be replaced or reconnected to IMD 16.

FIG. 4 is a functional block diagram illustrating an example configuration of external programmer 24 that facilitates user communication with IMD 16. As shown in FIG. 4, programmer 24 may include a processor 100, memory 102, user interface 104, telemetry module 106, power source 108, and lead diagnostic module 98. Programmer 24 may be a dedicated hardware device with dedicated software for programming of IMD 16. Alternatively, programmer 24 may be an off-the-shelf computing device running an application that enables programmer 24 to program IMD 16.

A user may use programmer 24 to select therapy programs (e.g., sets of stimulation parameters), generate new therapy programs, modify therapy programs through individual or global adjustments or transmit the new programs to a medical device, such as IMD 16 (FIG. 1). The clinician may interact with programmer 24 via user interface 104, which may include display to present graphical user interface to a user, and a keypad or another mechanism for receiving input from a user. In addition, the user may receive an alert or notification from IMD 16 indicating that IMD 16 has diagnosed a lead connection problem or a lead fracture, via programmer 24.

Processor 100 can take the form one or more microprocessors, DSPs, ASICs, FPGAs, programmable logic circuitry, or the like, and the functions attributed to processor 100 herein may be embodied as hardware, firmware, software or any combination thereof. Memory 102 may store instructions that cause processor 100 to provide the functionality ascribed to programmer 24 herein, and information used by processor 100 to provide the functionality ascribed to programmer 24 herein. Memory 102 may include any fixed or removable magnetic, optical, or electrical media, such as RAM, ROM, CD-ROM, hard or floppy magnetic disks, EEPROM, or the like. Memory 102 may also include a removable memory portion that may be used to provide memory updates or increases in memory capacities. A removable memory may also allow patient data to be easily transferred to another computing device, or to be removed before programmer 24 is used to program therapy for another patient.

Programmer 24 may communicate wirelessly with IMD 16, such as using RF communication or proximal inductive interaction. This wireless communication is possible through the use of telemetry module 106, which may be coupled to an internal antenna or an external antenna. An external antenna that is coupled to programmer 24 may correspond to the programming head that may be placed over heart 12, as described above with reference to FIG. 1. Telemetry module 106 may be similar to telemetry module 88 of IMD 16 (FIG. 4).

Telemetry module 106 may also be configured to communicate with another computing device via wireless communication techniques, or direct communication through a wired connection. Examples of local wireless communication techniques that may be employed to facilitate communication between programmer 24 and another computing device include RF communication according to the 802.11 or Bluetooth specification sets, infrared communication, e.g., according to the IrDA standard, or other standard or proprietary telemetry protocols. In this manner, other external devices may be capable of communicating with programmer 24 without needing to establish a secure wireless connection. An additional computing device in communication with programmer 24 may be a networked device such as a server capable of processing information retrieved from IMD 16.

In this manner, telemetry module 106 may receive a lead integrity diagnosis or lead integrity data from telemetry module 88 of IMD 16. The information may be automatically transmitted, or pushed, by IMD 16 when the diagnosis puts patient 14 at increased risk of harm. In addition, the alert may be a notification to a healthcare professional, e.g., a clinician or nurse, of the diagnosis and/or an instruction to patient 14 to seek medical assistance to remedy the problem with IMD 16 and leads 18, 20, or 22. In response to receiving the alert, user interface 104 may present the alert to the healthcare professional regarding diagnosis or present an instruction to patient 14 to seek medical treatment.

Lead diagnostic module 98 may, in one example, receive the diagnosis from IMD 16 to verify the diagnosis before presentation to the user. In another example, lead diagnosis module 98 may perform similar functions to that of lead diagnostic module 92 in IMD 16. In other words, lead diagnostic module 92 may receive transmitted lead integrity information, e.g., impedance measurements 83 and/or therapy episodes 85, from IMD 16 and generate the diagnosis within programmer 24. In this manner, lead diagnostic module 98 may cooperate with lead diagnostic module 92 of IMD 16 to diagnose any lead problems. Alternatively, either lead diagnostic module 92 of IMD 16 or lead diagnostic module 98 or programmer 24 may generate the diagnosis of a lead connection problem or a lead fracture. In other examples, a different external device, e.g., a network service, may generate the diagnosis.

User interface 104 may present the diagnosis of the lead connection problem or lead fracture to the user, e.g., a clinician, physician, other healthcare professional, or patient 14. A diagnosis of a lead connection problem may prevent unnecessary explantation of the medical lead that may have occurred without being able to differentiate between the two types of problems with leads 18, 20, and 22. User interface 104 may also allow the user to view the impedance measurements 83 used to generate the diagnosis and any other pertinent information. In some examples, user interface 104 may allow the user to view and/or change any of the thresholds or criteria used to automatically generate the diagnosis.

Upon receiving the alert or lead integrity information via user interface 104, the user may also interact with user interface 104 to cancel the alert, forward the alert, retrieve data regarding the diagnosis (e.g., impedance measurements 83), modify one or more instructions or criteria defining how the diagnosis is made, or conduct any other action related to the treatment of patient 14. In some examples, the clinician may be able to review raw data to diagnose any other problems with patient 14. User interface 104 may even suggest treatment along with the alert, e.g., alternative sensing or therapy configurations or drugs or doses to deliver until the lead problem can be fixed. User interface 104 may also allow the user to specify the type and timing of alerts based upon the severity or criticality of the diagnosis.

In some examples, processor 100 of programmer 24 and/or one or more processors of one or more networked computers may perform all or a portion of the techniques described herein with respect to processor 80 and IMD 16. For example, processor 100 and/or lead diagnostic module 98 within programmer 24 may analyze measured lead impedances to diagnose between a lead connection problem or a lead fracture problem.

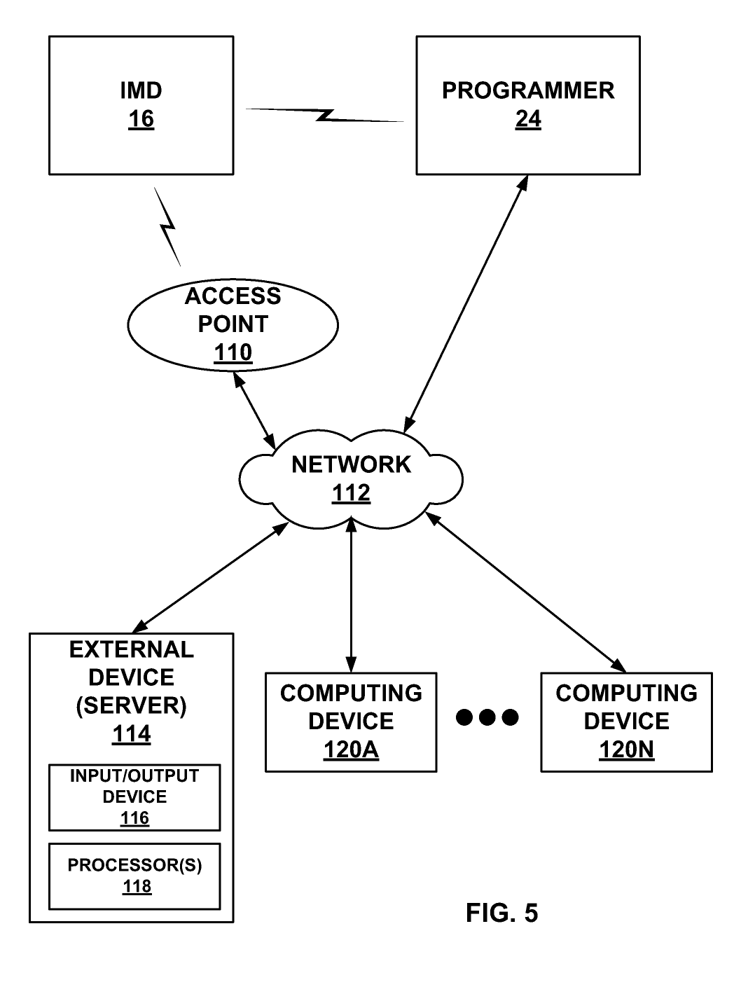

FIG. 5 is a block diagram illustrating an example system that includes an external device, such as a server 114, and one or more computing devices 120A-120N, that are coupled to the IMD 16 and programmer 24 shown in FIG. 1 via a network 112. Network 112 may be used to transmit a diagnosis of a lead connection or lead fracture (or unprocessed data) from IMD 16 to another external computing device. In this example, IMD 16 may use its telemetry module 88 to communicate with programmer 24 via a first wireless connection, and to communication with an access point 110 via a second wireless connection. In the example of FIG. 5, access point 110, programmer 24, server 114, and computing devices 120A-120N are interconnected, and able to communicate with each other, through network 112. In some cases, one or more of access point 110, programmer 24, server 114, and computing devices 120A-120N may be coupled to network 112 through one or more wireless connections. IMD 16, programmer 24, server 114, and computing devices 120A-120N may each comprise one or more processors, such as one or more microprocessors, DSPs, ASICs, FPGAs, programmable logic circuitry, or the like, that may perform various functions and operations, such as those described herein.

Access point 110 may comprise a device that connects to network 112 via any of a variety of connections, such as telephone dial-up, digital subscriber line (DSL), or cable modem connections. In other examples, access point 110 may be coupled to network 112 through different forms of connections, including wired or wireless connections. In some examples, access point 110 may be co-located with patient 14 and may comprise one or more programming units and/or computing devices (e.g., one or more monitoring units) that may perform various functions and operations described herein. For example, access point 110 may include a home-monitoring unit that is co-located with patient 14 and that may monitor the activity of IMD 16. In some examples, server 114 or computing devices 120 may control or perform any of the various functions or operations described herein, e.g., generate a heart failure risk score based on the patient metric comparisons or create patient metrics from the raw metric data.

In some cases, server 114 may be configured to provide a secure storage site for archival of lead integrity data (e.g., raw data and/or diagnoses) that has been collected and generated from IMD 16 and/or programmer 24. Network 112 may comprise a local area network, wide area network, or global network, such as the Internet. In some cases, programmer 24 or server 114 may assemble sensing integrity information in web pages or other documents for viewing by and trained professionals, such as clinicians, via viewing terminals associated with computing devices 120. The system of FIG. 5 may be implemented, in some aspects, with general network technology and functionality similar to that provided by the Medtronic CareLink® Network developed by Medtronic, Inc., of Minneapolis, Minn.

In the manner of FIG. 5, computing device 120A or programmer 24, for example, may be remote computing devices or external devices that receive and present a lead integrity diagnosis from IMDs of one or more patients. In some examples, each IMD may transmit the measured impedances 83, therapy episodes 85, or other data so that computing device 120A, external device 114, or remote programmer 24 may process the data to generate a diagnosis of lead connection problems or lead fractures. In other examples, IMD may transmit the finished diagnosis of a lead fracture or lead connection problem. Therefore, a clinician may be able to remotely treat patient 14. This method may useful for healthcare professionals making house calls, serving patients within a nursing home, serving patients living far from a medical facility, or any other circumstance in which a professional treats many patients.

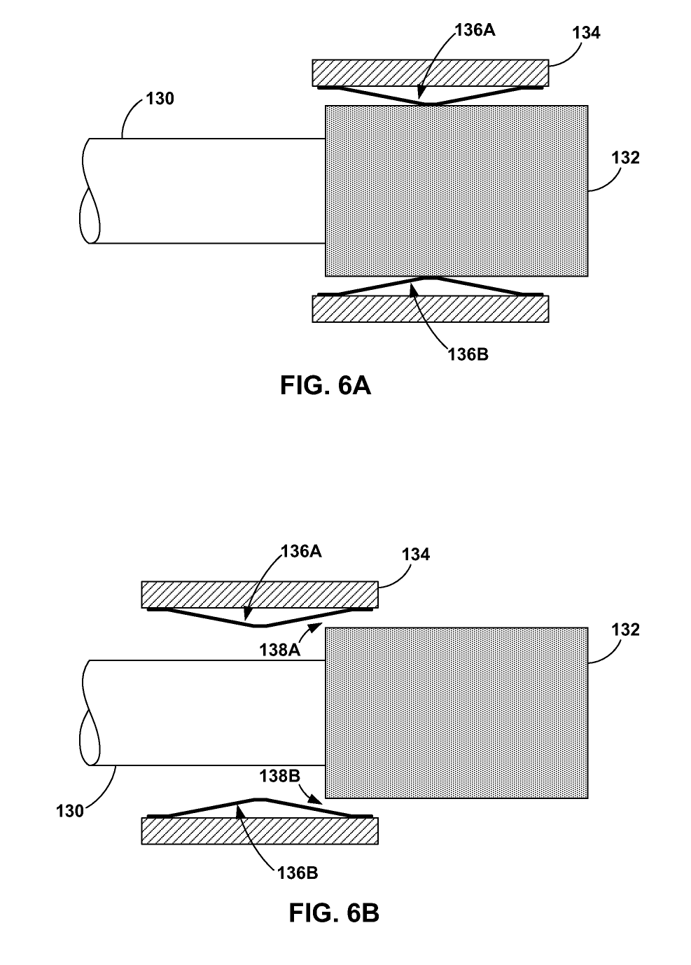

FIGS. 6A and 6B are conceptual illustrations of example complete and incomplete connections of lead connector 132 within a header of IMD 16. As described above, an incomplete connection, or connection problem, may be more subtle than a complete disconnection between a lead pin and IMD 16. For example, a connection problem may also include lead pin 130 only partially inserted into header connector 134 or a less than full tightening of the set screw such that lead pin 130 does not make a complete electrical connection with header connector 134 of IMD 16. FIGS. 6A and 6B only illustrate a portion of a lead, e.g., leads 18, 20, and 22, that would be within header 34 of IMD 16, for example. As shown in FIG. 6A, the lead has a complete connection to the header that would allow for normal operation of the lead. Lead connector 132 is attached to lead pin 130. In some examples, lead connector 132 may be a ring electrode with an electrically conductive material. Lead pin 130 may be fixed within the header with one or more set screws during connection of the lead with the IMD.

Header connector 134 is electrically coupled to IMD 16 and may surround at least a portion of lead connector 132. Similar to lead connector 132, header connector 134 may be a ring electrode in some examples. Springs 136A and 136B (collectively “springs 136”) are mounted to the inside of header connector 134 and configured to make physical contact with lead connector 132 to electrically couple the lead with the IMD. In the example of FIG. 6A the pin of the lead has been positioned completely within the header such that lead connector 132 is contacting springs 126. In this complete connection, electrical current may flow freely between header connector 134 and lead connector 132 such that no increased impedances are detected in the lead.

In contrast, FIG. 6B illustrates incomplete insertion of the lead pin within header 34 of IMD 16. As shown in FIG. 6B, lead connector 132 is not inserted completely within header connector 134. Gaps 138A and 138B are shown between springs 136 and lead connector 132. Neither of springs 136 directly contacts lead connector 132, so the impedance measured with this connection may be an increased impedance that is higher than a baseline impedance value. Even though no contact is made between springs 136 and lead connector 132, electrical current may still flow between lead connector 132 and header connector 134. However, the impedance between the structures may be measurably higher than if springs 136 contacted lead connector 132. The incomplete connection of lead connector 132 and header connector 134 may be remedied by disengaging the set screws in lead pin 130, sliding lead connector 132 fully within header connector 134, and re-engaging the set screws. Therefore, a diagnosis of a lead connection problem may allow the connection problem to be solved without explanting the incompletely connected lead. In another example, the connection may only be sufficient to cause an increase in impedance instead of also causing oversensing. The lead may continue to be used and monitored until oversensing is also detected, indicating that the connection problem may need to be fixed.

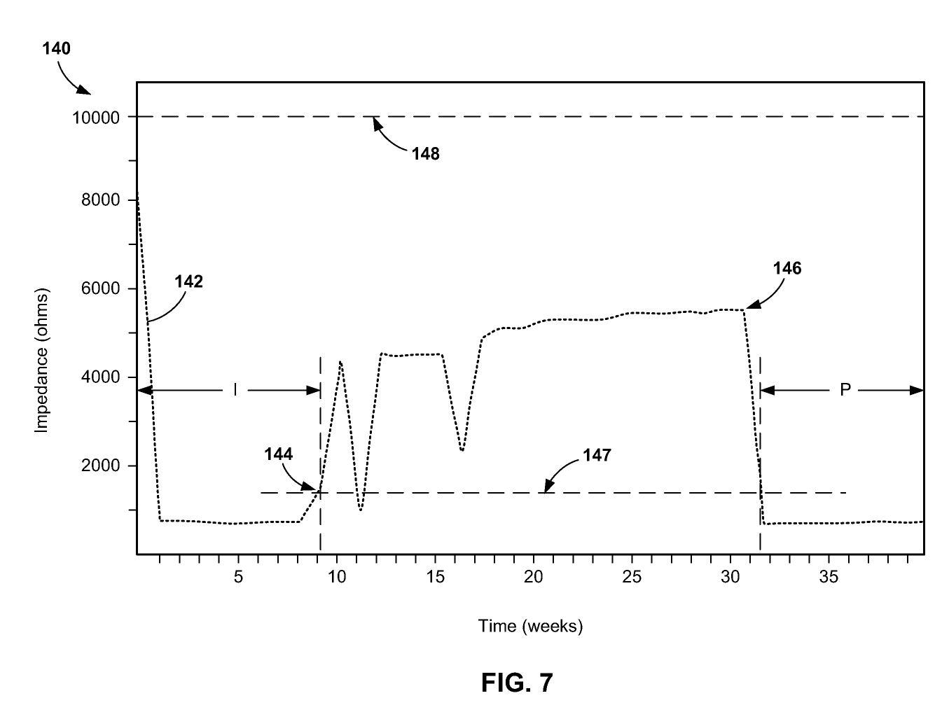

FIG. 7 illustrates example graph 140 of impedance values 142 measured over time from a lead diagnosed with a lead connection problem. As shown in FIG. 7, impedance values 142 in ohms are plotted versus time in weeks in graph 140. Impedance values 142 may be measured once daily, but more or less frequent impedance measurements may be performed by IMD 16. Impedance values 142 may be an example of impedance measurements 83 stored in memory 82 of IMD 16.

Impedance values 142 initially started at an elevated level immediately after implantation of the lead, but decreased to the normal operating impedance range of approximately 800 ohms between the first and eighth week after connecting the lead to IMD 16, e.g., post implant. An increased impedance value 144, e.g., an abrupt rise in impedance, is detected when a measured impedance magnitude is greater than magnitude threshold 147. As described herein, magnitude threshold 147 is a threshold above a baseline impedance value, which may be an average of previously measured impedance values, and may represent the normal operating impedance range. In other examples, magnitude threshold may be a constant magnitude irrelevant of the baseline.

In the example of graph 140, the baseline impedance value is approximately 800 ohms. Increased impedance 144 is detected when the impedance exceeds magnitude threshold 147, e.g., 1150 ohms. This increased impedance value 144 is greater than the magnitude threshold of 350 ohms above the baseline impedance value of approximately 800 ohms, e.g., approximately 1150 ohms. Increased impedance value 144 may be characterized as an abrupt rise in impedance because it exceeds a moving average by at least a threshold amount.

Time period I indicates that the time between connection of the lead to IMD 16 and increased impedance 144 is approximately 70 days. Then, impedance values 142 remain increased for many weeks at a magnitude over 5,000 ohms until impedance values 142 reach maximum impedance value 146, approximately 5,700 ohms. Impedance values 142 then return to the baseline, e.g., the average of impedance values measured prior to the detection of the increased impedance 144, such as from time period I, for an extended period of time (approximately 70 days) indicated by time period P. Although not necessary since a baseline impedance value does not need to be equivalent to a prior baseline, impedance values 142 during time period P are below magnitude threshold 147.

In some examples in which a lead diagnostic module 92, 98 uses an average of measured impedance values as a baseline impedance, the lead diagnostic module may suspend updating the average upon detection of increased impedance value 144. The average at that point may be stored within a memory, e.g., memory 82. The stored average may be used to detect a return to baseline impedance. In some examples, a threshold impedance value for detecting a return to baseline impedance may be set above the stored average, e.g., an absolute number or percentage of the stored average above the stored average.

In addition, the baseline impedance value may also be updated or changed, before or after any detected increase in impedance. For example, the baseline impedance value may be updated after the abrupt rise in impedance if the most recent impedance values are determined to be within a normal operating range and substantially different from the previous baseline values prior to the abrupt rise in impedance. In other words, the baseline impedance value may be updated or changed over time to compensate for normal variations and/or drift in measured impedance values not related to connector problems or lead fractures. Updating the baseline impedance value may help to avoid a false positive diagnosis of a lead fracture, for example. Baseline impedance values may be updated periodically, e.g., daily, weekly, or monthly, based on recent impedance measurements.

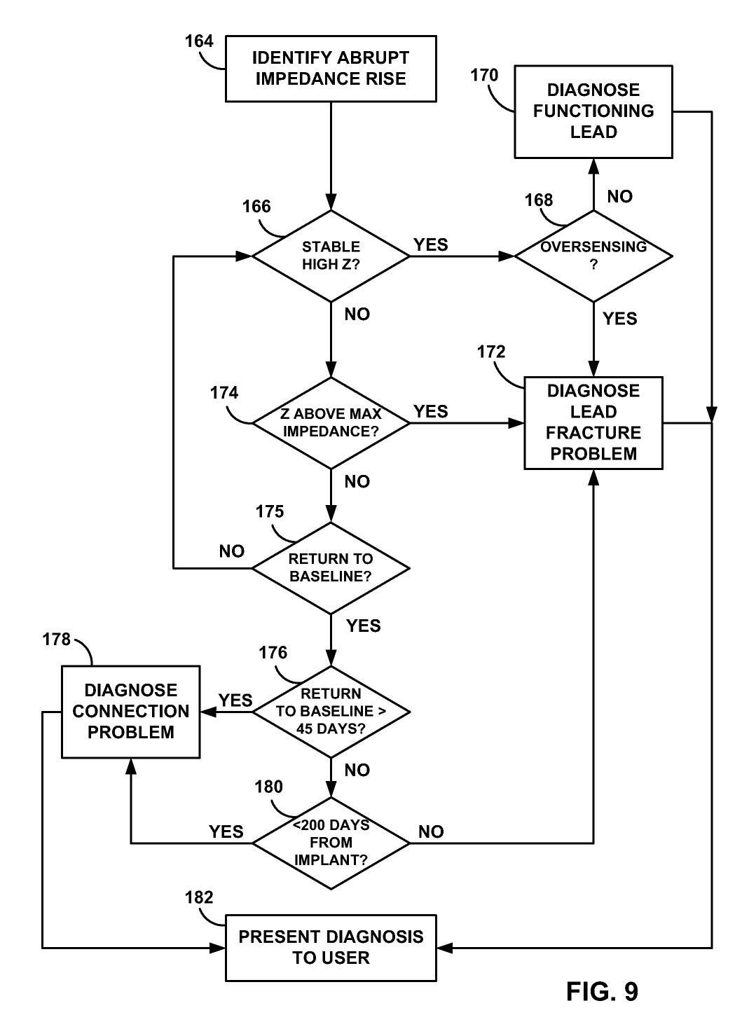

According to the example criteria described in greater detail below with respect to FIG. 9, impedance values 142 of graph 140 indicate a diagnosis of a lead connection problem. Maximum impedance value 146 is below maximum impedance threshold 148, e.g., 10,000 ohms, so a lead fracture is not indicated using these criteria. In addition, the return to baseline impedance values for the extended period of 70 days, as indicated by time period P, is greater than a duration threshold of approximately 45 days. Since impedance values of lead fractures generally would not return to the baseline impedance value for a time greater than the duration threshold, graph 140 indicates that the lead is not completely connected to header 34 of IMD 16. Moreover, increased impedance value 144 occurred within the duration threshold of the connection to increased impedance interval, e.g., 200 days, which also indicates a lead connection problem. The lead of graph 140 may not have a fracture and may continue to be used in the patient once the connection problem is resolved.

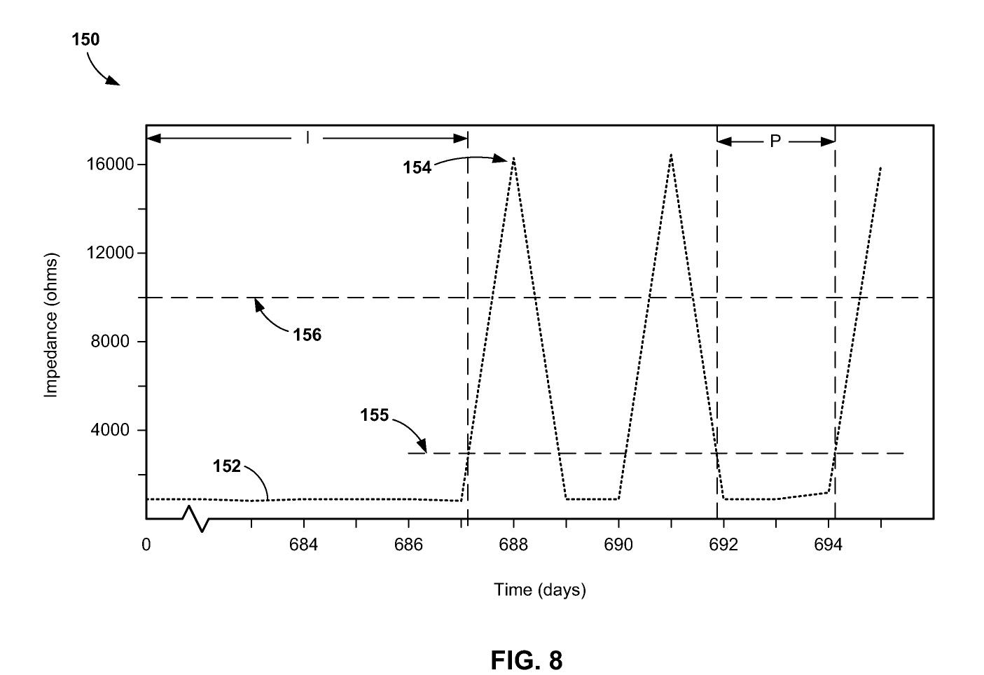

FIG. 8 illustrates an example graph 150 of impedance values 152 measured over time from a lead diagnosed with a lead fracture, in contrast to the lead connection problem illustrated by FIG. 7. As shown in FIG. 8, impedance values 152 in ohms are plotted versus time in days in graph 150. Impedance values 152 may be measured once daily, but more or less frequent impedance measurements may be performed by IMD 16. Impedance values 152 may be similar to impedance measurements 83 stored in memory 82 of IMD 16.

Impedance values 152 are shown at approximately 500 ohms after connection of the lead with IMD 16, during time period I. Impedance values measured during time period I may also be used to calculate the baseline impedance value which may be used to determine when an increased impedance value, or abrupt rise, occurs. An increased impedance value may be any impedance measured over magnitude threshold 147, which may be set above a baseline impedance value or set to a constant value. An increase in impedance values 152 occurs at 688 days from connection of the lead, as indicated by time period I. The increase in impedance values 152 includes maximum impedance value 154, shown at approximately 16,000 ohms. Maximum impedance value 154 is greater than magnitude threshold 155 and also greater than maximum impedance threshold 156, e.g., 10,000 ohms. Since maximum impedance value 154 is greater than magnitude threshold 155, e.g., 350 ohms above the baseline impedance value, impedance value 154 may also be an abrupt rise in impedance. As described herein, the abrupt rise in impedance may be determined when an impedance value increases more than the magnitude threshold, and sometimes also within a predetermined period of time. Magnitude threshold 155 may be set at a predetermined value above the baseline impedance value. In the example of FIG. 8, an increase in impedance greater than 850 ohms, e.g., a baseline impedance value of 500 ohms and a magnitude threshold of another 350 ohms, may be determined as an abrupt rise. Impedance values 152 then return to the baseline impedance value at day 692, but then impedance values 152 increase again after time period P of only 2 days.

According to the example criteria of FIG. 9, impedance values 152 of graph 150 indicate a diagnosis of a lead fracture. Impedance values 152 become greater than maximum impedance threshold 156, so a lead fracture is automatically indicated when threshold 156 is crossed. Maximum impedance threshold 156 is set at 10,000 ohms in the example of FIG. 9. Indeed, maximum impedance value 154 is shown at approximately 16,000 ohms, well above threshold 156. Moreover, an abrupt rise in impedance values 152 occurred at approximately 687 days after the lead was connected to IMD 16. This interval of 687 days since the connection is greater than a duration threshold that indicates a lead connection problem is unlikely, e.g., greater than 200 days. In other words, no increases above magnitude threshold 155 for a time greater than the duration threshold may indicate that the connection between the lead and IMD 16 is sufficient. In addition, there is no return to the baseline impedance value, e.g., the average of previous impedance values, similar to the one described above in FIG. 7. Time period P is only approximately 2 days, which is shorter than the duration threshold required to diagnose the problem as a lead connection problem, e.g., 45 days in some examples. Therefore, a lead exhibiting impedance values similar to impedance values 152 may have a fracture.

FIG. 9 is a flow diagram of an example method for diagnosing lead fractures and lead connection problems. FIG. 9 will be described with lead diagnostic module 92 of IMD 16 diagnosing lead connection problems or lead fractures. However, the techniques of FIG. 9 may also be performed with lead diagnostic module 98 of programmer 24, an external device on a network such as server 114 of FIG. 5, or any other computing device. In this manner, the techniques of FIG. 9 may be performed in real-time as impedance measurements are performed on a lead or retroactively over stored impedance values. Also, lead 18 will be used for example diagnosis, but any of leads 18, 20, and 22, or other leads described herein may be diagnosed when necessary. IMD 16 may first measure lead impedances, identify therapy episodes, collect oversensing information, and/or determine other lead characteristics, e.g., lead integrity information, with sensing module 86 and transmit this lead integrity information to lead diagnostic module 92.

Lead diagnostic module 92 may, after measuring lead impedance or beginning to analyze prior impedance measurements, determine if the increased impedance value is an abrupt rise in impedance (164). As described herein, an abrupt rise in impedance may be an impedance value that rises more than 350 ohms or 60 percent above the baseline impedance value. This increased impedance value may need to occur within a predetermined period of time, e.g., 24 hours, in some examples to be identified as an increased impedance value. Alternatively, as described above, lead diagnostic module 92 may be required to identify two or more increased impedance values before determining that an abrupt rise in impedance has occurred.

Once an increased impedance value is identified, lead diagnostic module 92 determines if increased impedance values indicate a stable high impedance value (166). If lead diagnostic module 92 determines there is a stable high impedance level (“YES” branch of block 166), lead diagnostic module 92 continues with the oversensing analysis of block 168. Oversensing may be determined with a variety of methods. For example, lead diagnostic module 92 may use the number of shocks delivered to patient 14. In other examples, oversensing can be determined based on the number of non-sustained tachyarrhythmias or short intervals stored in oversensing episodes 87. In any event, oversensing occurs when either abnormal cardiac signals or noise is detected from lead 18 that causes IMD 16 to measure a greater frequency of heart beats than is actually occurring. Although any oversensing episodes 87 or therapy episodes 85 may be used to detect oversensing, lead diagnostic module 92 may only use those episodes that occur shortly before the identified increased impedance value, e.g., one day or one week, and after the increased impedance value. If oversensing is detected by lead diagnostic module 92 (“YES” branch of block 168), then lead diagnostic module 92 diagnoses a lead fracture problem (172). If lead diagnostic module 92 does not detect any oversensing (“NO” branch of block 168), lead diagnostic module 92 diagnoses lead 18 as a functioning lead that may continue to be used for monitoring and therapy of patient 14 (170).

If lead diagnostic module 92 does not detect a stable high impedance level (“NO” branch of block 166), lead diagnostic module 92 determines if the increased impedance value is a very high impedance value (174). A very high impedance value may be an impedance value that is greater than the maximum impedance threshold. The maximum impedance threshold may be predetermined or varied according to system 10 circumstances, but the maximum impedance threshold may be set to an impedance magnitude above which are impedances typically only measured from fractured leads. If the increased impedance value exceeds the maximum impedance threshold (“YES” branch of block 174), lead diagnostic module 92 diagnoses a lead fracture (172).

If lead diagnostic module 92 determines that the increased impedance value is not a very high impedance value greater than the maximum impedance threshold (“NO” branch of block 174), then lead diagnostic module 92 determines if the measured impedance values have returned to a baseline impedance value, e.g., average of previously measured impedance values indicative of a normal operating impedance value (175). If the impedance values have not returned to baseline (“NO” branch of block 175), then lead detection module 92 continues to determine if the high impedance values are stable (166).

If the impedance values have returned to baseline (“YES” branch of block 175), lead detection module 92 determines whether measured impedance values again abruptly rise within an interval of a predetermined duration from the return to baseline, i.e., determines whether the measured impedance remain at or near the baseline for at least the predetermined duration threshold. If lead diagnostic module 92 determines that there was a return to the baseline impedance value for more than the duration threshold (e.g., 45 days) after the increased impedance value (e.g., an abrupt rise in impedance) was detected (“YES” branch of block 176), then lead diagnostic module 92 diagnoses a lead connection problem between lead 18 and IMD 16. If lead diagnostic module 92 determines that any return to baseline after the increased impedance value is less than the duration threshold of 45 days (“NO” branch of block 176), but lead diagnostic module 92 determines that the increased impedance value occurred less than an interval with a predetermined duration, e.g., of 200 days, from connection of lead 18 to IMD 16 (“YES” branch of block 180), then lead diagnostic module 92 also diagnoses a lead connection problem. If the increased impedance value occurred more than the duration threshold, e.g., 200 days, after connection of lead 18 with IMD 16 (“NO” branch of block 180), then lead diagnostic module 92 diagnoses a lead fracture (172).

According to the criteria provided in FIG. 9, lead diagnostic module 92 may diagnose a lead problem as a lead connection problem, a lead fracture, or even a functioning lead after detecting an increased impedance value. After making the diagnosis, lead diagnostic module 92 may transmit the diagnosis to programmer 24 for presentation of the diagnosis to the user via user interface 104 of programmer 24 (182). The presentation of the diagnosis may provide steps the clinician can take to remedy the problem and/or configure IMD 16 before reconnecting lead 18 or replacing lead 18. In some examples, user interface 104 may allow the user to review impedance measurements 83, therapy episodes 85, oversensing episodes 87, or connection dates used by lead diagnostic module 92 to generate the diagnosis. User interface 104 then allow the user to restart therapy, adjust therapy parameters, or address other problems as desired by the user.

Diagnosis of the lead connection problem, lead fracture, or functioning lead by lead diagnostic module 92 may differ from the example of FIG. 9 in one or more aspects. In some cases, for example, a lead may still be diagnosed with a connection problem if the measured impedance exceeds the stable high impedance threshold of block 166 and no oversensing was detected in block 168. Before diagnosing the lead as a functioning lead in block 170, lead diagnostic module 92 may evaluate whether there was a return to baseline greater than 45 days (block 176) and whether the increased impedance value occurred less than 200 days from connection of lead 18 to IMD 16 (block 180). If either of these conditions are satisfied, lead diagnostic module 92 may diagnose the lead as having a lead connection problem. If neither of these conditions are satisfied, lead diagnostic module 92 may still diagnose the lead as functioning (170).

In another example, lead diagnostic module 92 may employ a normal impedance threshold. If the increased impedance value is greater than the normal impedance threshold, lead diagnostic module 92 may be prevented from diagnosing the lead as a normal functioning lead in block 170. Lead diagnostic module 92 may compare the increased impedance value to the normal impedance threshold prior to block 170. If the increased impedance value is greater than the normal impedance threshold, then lead diagnostic module 92 may further compare the increased impedance value to other criteria before diagnosis, e.g., re-enter the flow diagram at block 174. The normal impedance threshold may be set between the magnitude threshold above baseline, e.g., 350 ohms above baseline, and the maximum impedance threshold. For example, the normal impedance threshold may be set between approximately 2,000 ohms and 2,500 ohms, or at a certain magnitude above the baseline impedance value.

Since the diagnostic technique described herein is not intended to be limited to the flow diagram of FIG. 9, IMD 16, programmer 24, or any other device may implement the diagnostic criteria in other methods. For example, lead diagnostic module 92 may simply have a list of each criteria necessary for the diagnosis to be a functioning lead, lead fracture, and lead connection problem, and generate the appropriate diagnosis when the criteria for one diagnosis is fulfilled. In one example, lead diagnostic module 92 may simply diagnose a lead connection problem after detecting an abrupt rise in the impedance value, the impedance value is below a maximum impedance threshold, and the impedance values return to the baseline impedance value for at least 45 days. Therefore, the diagnosis does not need to be sequential as described in FIG. 9.

The techniques described herein may, for example, allow an IMD, a programmer, a networked device, or other external device to diagnose problems with a lead to avoid unnecessary procedures. Since high impedance measurements of a lead are typically associated with lead fractures, clinicians may immediately explant the lead because it is difficult to determine if there is another non-fracture problem instead. However, automatically diagnosing the actual problem with the lead as described herein may allow differentiation between incomplete lead connections and fractured leads. The clinician may thus only explant leads that are diagnosed with a lead fracture and require replacement. Leads diagnosed with a lead connection problem may be easily fixed by the clinician with a simple surgical procedure to expose the header of the IMD and correctly and completely connect the lead pin with the header. This diagnosis technique thus reduces unnecessary pain to the patient associated with removing a functional lead, potential damage to sensitive tissue with implanting a new lead, added healing time before therapy can begin again, and the cost of unneeded explantations. The techniques described herein may also allow for remote diagnosis of leads or an alert to patients in order to expedite the repair of any lead problem.

Various examples have been described that include automatic diagnosis of lead connection problems and lead fractures. These examples include techniques for diagnosing incomplete lead connections with an IMD and lead fractures. In addition, an alert of the diagnosis may be remotely delivered to a healthcare professional for earlier treatment and repair of implanted components. Any combination of diagnosis and notification of diagnosis is contemplated. These and other examples are within the scope of the following claims.

1. A method comprising:
measuring a plurality of impedance values of an implantable medical lead;
comparing each of the impedance values to a threshold;
identifying at least one of the plurality of impedance values greater than the threshold as an increased impedance value;
determining a timing of the increased impedance value; and
automatically selecting between a diagnosis of a lead fracture or a diagnosis of a lead connection problem based on the timing of the increased impedance value.

2. The method of claim 1, wherein the threshold comprises a threshold set above a baseline impedance value.

3. The method of claim 2, wherein the threshold is at least one of approximately 350 ohms or 60 percent greater than the baseline impedance value.

4. The method of claim 1, wherein determining the timing of the increased impedance value further comprises determining whether the increased impedance value occurred within a predefined interval.

5. The method of claim 4, wherein determining whether the increased impedance value occurred within the predefined interval comprises determining whether the increased impedance value occurred within the predefined interval from a connection of the medical lead to an implantable medical device, and wherein selecting between a diagnosis of a lead fracture or a diagnosis of a lead connection problem comprises selecting the diagnosis of the lead connection problem when the increased impedance value occurred within the predefined interval.

6. The method of claim 5, wherein the predefined interval is approximately 200 days.

7. The method of claim 4, wherein determining whether the increased impedance value occurred within the predefined interval comprises determining whether the increased impedance value occurred within the predefined interval from a return to baseline impedance values, and wherein selecting between a diagnosis of a lead fracture or a diagnosis of a lead connection problem comprises selecting the diagnosis of the lead connection problem when the increased impedance value occurred outside of the predefined interval.

8. The method of claim 7, wherein the predefined interval is approximately 45 days.

9. The method of claim 1, further comprising determining that a maximum impedance value of the plurality of impedance values is greater than a maximum impedance threshold, wherein the diagnosis of the lead fracture is automatically selected upon the determination.

10. The method of claim 9, wherein the maximum impedance threshold is approximately 10,000 ohms.

11. The method of claim 1, further comprising:
comparing measured impedances subsequent to the increased impedance value to a stable high impedance threshold;
determining that a stable high impedance exists when consecutive ones of the measured impedances subsequent to the increased impedance value exceed the stable high impedance threshold;
determining oversensing from the medical lead; and
automatically selecting the diagnosis of the lead fracture upon the determination of the stable high impedance level and the oversensing.

12. The method of claim 1, wherein the diagnosis of the lead connection problem is automatically selected upon determining the timing of the increased impedance value is within a threshold period of time from connecting the medical lead to an implantable medical device.

13. The method of claim 1, further comprising increasing an impedance measuring frequency in response to identifying at least one of the impedance values greater than the threshold.

14. A system comprising:
an implantable medical device that measures a plurality of impedance values of an implantable medical lead coupled to the implantable medical device; and
a lead diagnostic module configured to:
compare each of the impedance values to a threshold;
identify at least one of the plurality of impedance values greater than the threshold as an increased impedance value;
determine a timing of the increased impedance value; and
automatically select between a diagnosis of a lead fracture or a diagnosis of a lead connection problem based on the timing of the increased impedance value.

15. The system of claim 14, wherein the threshold comprises a threshold set above a baseline impedance value.

16. The system of claim 15, wherein the increased impedance value is at least one of approximately 350 ohms or 60 percent greater than the baseline impedance value.

17. The system of claim 14, wherein the lead diagnostic module determines the timing of the increased impedance value by determining whether the increased impedance value occurred within a predefined interval.

18. The system of claim 17, wherein the lead diagnostic module determines whether the increased impedance value occurred within the predefined interval from a connection of the medical lead to an implantable medical device, and wherein the lead diagnostic module selects the lead connection problem when the increased impedance value occurred within the predefined interval.

19. The system of claim 17, wherein the lead diagnostic module determines whether the increased impedance value occurred within the predefined interval from a return to baseline impedance values, and wherein the lead diagnostic module selects the lead connection problem when the increased impedance value occurred outside of the predefined interval.

20. The system of claim 14, wherein the lead diagnostic module is configured to:
determine a maximum impedance value of the plurality of impedance values greater than a maximum impedance threshold; and
automatically select the diagnosis of the lead fracture upon the determination.

21. The system of claim 20, wherein the maximum impedance threshold is approximately 10,000 ohms.

22. The system of claim 14, wherein the lead diagnostic module is configured to:
compare measured impedances subsequent to the increased impedance value to a stable high impedance threshold;
determine that a stable high impedance exists when consecutive ones of the measured impedances subsequent to the increased impedance value exceed the stable high impedance threshold;
determine oversensing from the medical lead; and
automatically select the diagnosis of the lead fracture upon the determination of the stable high impedance level and the noise oversensing.

23. The system of claim 22, wherein the stable high impedance threshold comprises a percentage of a maximum measured impedance value.

24. The system of claim 14, wherein the lead diagnostic module automatically selects the diagnosis of the lead connection problem upon determining the timing of the increased impedance value is within a threshold period of time from connecting the medical lead to an implantable medical device.

25. The system of claim 14, wherein the lead diagnostic module is configured to increase an impedance measuring frequency in response to identifying one of the impedance values greater than the threshold.

26. The system of claim 14, wherein the implantable medical device comprises the lead diagnostic module.

27. A system comprising:
means for measuring a plurality of impedance values of an implantable medical lead;
means for comparing each of the impedance values to a threshold;
means for identifying at least one of the plurality of impedance values greater than the threshold as an increased impedance value;
means for determining a timing of the increased impedance value; and
means for automatically selecting between a diagnosis of a lead fracture or a diagnosis of a lead connection problem based on the timing of the increased impedance value.

28. The system of claim 27, further comprising means for presenting the automatically selected diagnosis to a user, wherein:
the means for determining the timing determines when the increased impedance value is an abrupt rise in impedance magnitude over a baseline impedance value within a predetermined time period;
the means for automatically selecting between a diagnosis of a lead fracture or a diagnosis of a lead connection problem automatically selects the diagnosis of the lead connection problem when the increased impedance value is the abrupt rise and at least one of the timing of the increased impedance value occurs less than approximately 200 days from a connection of the medical lead to an implantable medical device or a return to baseline impedance values after a previous increased impedance value occurs for greater than approximately 45 days; and
the means for automatically selecting between a diagnosis of a lead fracture or a diagnosis of a lead connection problem automatically selects the diagnosis of the lead fracture when at least one of a maximum impedance value of the plurality of impedance values is greater than a maximum impedance threshold or oversensing is determined from the medical lead.
